(19) United States
(12) Patent Application Publication
(10) Pub. No.: US 2007/0010004 A1
(43) Pub. Date: Jan. 11, 2007

(54) Micellar systems

(76) Inventors: Sean D. Monahan, Mazomanie, WI (US); Vladimir G. Budker, Middleton, WI (US); Tatyana Budker, Middleton, WI (US); Jon A. Wolff, Madison, WI (US); Paul M. Slattum, Salt Lake City, UT (US); James E. Hagstrom, Middleton, WI (US)

Correspondence Address: MIRUS CORPORATION, 505 SOUTH ROSA RD, MADISON, WI, 53719, US

(21) Appl. No.: 11/479,587

(22) Filed: Jun. 30, 2006

Related U.S. Application Data
(63) Continuation-in-part of application No. 10/627,247, filed on Jul. 25, 2003, now Pat. No. 7,091,041.
Application No. 10/627,247 is a division of application No. 10/081,461, filed on Feb. 21, 2002, now Pat. No. 6,673,612.
Application No. 10/081,461 is a continuation-in-part of application No. 09/354,957, filed on Jul. 16, 1999, now Pat. No. 6,429,200.
(60) Provisional application No. 60/093,321, filed on Jul. 20, 1998.

(51) Int. Cl. C12N 1/08; C07H 21/00

(52) U.S. Cl. 435/270; 536/25.4

(57) ABSTRACT

Methods are described for modifying nucleic acids to facilitate delivery of the nucleic acids to cells. Compounds which interact with of modify nucleic acids are interacted with the nucleic acids within reverse micelles.

### CROSS-REFERENCE TO RELATED APPLICATIONS

This application is a continuation-in-part of Application Ser. No. 10/627,247, filed Jul. 25, 2003, which is a divisional of application Ser. No. 10/081,461; filed Feb. 21, 2002, issued ad U.S. Pat. No. 6,673,612, which is a continuation-in-part of application Ser. No. 09/354,957, filed Jul. 16, 1999, issued as U.S. Pat. No. 6,429,200, which claims the benefit of U.S. Provisional Application No. 60/093,321, filed Jul. 17, 1998.

### FIELD OF THE INVENTION

The invention generally relates to micellar systems for use in biologic systems. More particularly, a process is provided for the use of reverse micelles for the covalent modification of nucleic acids, the preparation of nucleic acid complexes, and for the delivery of nucleic acids and genes to cells.

### BACKGROUND

Biologically active compounds such as proteins, enzymes, and nucleic acids have been delivered to the cells using amphipathic compounds that contain both hydrophobic and hydrophilic domains. Typically these amphipathic compounds are organized into vesicular structures such as liposomes, micellar, or inverse micellar structures. Liposomes can contain an aqueous volume that is entirely enclosed by a membrane composed of lipid molecules (usually phospholipids) (R. C. New, p. 1, chapter 1, “Introduction” in Liposomes: A Practical Approach, ed. R. C. New IRL Press at Oxford University Press, Oxford, 1990). Micelles and inverse micelles are microscopic vesicles that contain amphipathic molecules but do not contain an aqueous volume that is entirely enclosed by a membrane. In micelles the hydrophilic part of the amphipathic compound is on the outside (on the surface of the vesicle) whereas in inverse micelles the hydrophobic part of the amphipathic compound is on the outside. The inverse micelles thus contain a polar core that can solubilize both water and macromolecules within the inverse micelle. As the volume of the core aqueous pool increases the aqueous environment begins to match the physical and chemical characteristics of bulk water. The resulting inverse micelle can be referred to as a microemulsion of water in oil (Schelly, Z. A. Current Opinion in Colloid and Interface Science, 37-41, 1997; Castro, M. J. M., Cabral, J. M. S. Biotech. Adv. 6, 151-167, 1988).

Microemulsions are isotropic, thermodynamically stable solutions in which substantial amounts of two immiscible liquids (water and oil) are brought into a single phase due to a surfactant or mixture of surfactants. The spontaneously formed colloidal particles are globular droplets of the minor solvent, surrounded by a monolayer of surfactant molecules. The spontaneous curvature, H0 of the surfactant monolayer at the oil/water interface dictates the phase behavior and microstructure of the vesicle. Hydrophilic surfactants produce oil in water (O/W) microemulsions (H0>0), whereas lipophilic surfactants produce water in oil (W/O) microemulsions. When the hydrophile-lipophile properties of the surfactant monolayer at the water/oil interface are balanced bicontinuous-type microemulsions are formed (H0=0).

Positively-charged, neutral, and negatively-charged liposomes have been used to deliver nucleic acids to cells. For example, plasmid DNA expression in the liver has been achieved via liposomes delivered by tail vein or intraportal routes. Positively-charged micelles have also been used to package nucleic acids into complexes for the delivery of the nucleic acid to cells. Negatively-charged micelles have been used to condense DNA, however they have not been used for the delivery of nucleic acids to cells (Imre, V. E., Luisi, P. L. Biochemical and Biophysical Research Communications, 107, 538-545, 1982). This is because the previous efforts relied upon the positive-charge of the micelles to provide a cross-bridge between the polyanionic nucleic acids and the polyanionic surfaces of the cells. Micelles that are not positively-charged, or that do not form a positively charged complex cannot perform this function. For example, a recent report demonstrated the use of a cationic detergent to compact DNA, resulting in the formation of a stable, negatively-charged particle (Blessing, T., Remy, J. S., Behr, J. P. Proc. Natl. Acad. Sci. USA, 95, 1427-1431, 1998). A cationic detergent containing a free thiol was utilized which allowed for an oxidative dimerization of the surfactant to the disulfide in the presence of DNA. However, as expected, the negatively-charged complex was not effective for transfection. Reverse (water in oil) micelles have also been used to make cell-like compartments for molecular evolution of nucleic acids (Tawfik, D. S. and Griffiths, A. D. Nature Biotechnology 16:652, 1998). In addition, Wolff et al. have developed a method for the preparation of DNA/amphipathic complexes including micelles in which at least one amphipathic compound layer that surrounds a non-aqueous core that contains a polyion such as a nucleic acid (Wolff, J., Budker, V., and Gurevich, V. U.S. Pat. No. 5,635,487).

Cleavable Micelles

A new area in micelle technology involves the use of cleavable surfactants to form the micelle. Surfactants containing an acetal linkage, azo-containing surfactants, elimination of an ammonium salt, quaternary hydrazonium surfactants, 2-alkoxy-N,N-dimethylamine N-oxides, and ester containing surfactants such as ester containing quaternary ammonium compounds and esters containing a sugar have been developed.

These cleavable surfactants within micelles are designed to decompose on exposure to strong acid, ultraviolet light, alkali, and heat. These conditions are very harsh and are not compatible with retention of biologic activity of biologic compounds such as proteins or nucleic acids. Thus, biologically active compounds have not been purified using reverse micelles containing cleavable surfactants.

Micelles and Reverse Micelles

Reverse micelles (water in oil microemulsions) are widely used as a host for biomolecules. Examples exist of both recovery of extracellular proteins from a culture broth and recovery of intracellular proteins. Although widely used, recovery of the biomolecules is difficult due to the stability of the formed micelle and due to incomplete recovery during the extraction process. Similarly, purification of DNA or other biomolecules from endotoxin and plasma is difficult to accomplish. One common method employing Triton results in incomplete separation of the DNA or biomolecules from the emulsion.

Reverse micelles have been widely used as a host for enzymatic reactions to take place. In many examples, enzymatic activity has been shown to increase with micelles, and has allowed enzymatic reactions to be conducted on water insoluble substrates. Additionally, enzymatic activity of whole cells entrapped in reverse micelles has been investigated (Gajjar L et al. Applied Biochemistry and Biotechnology, 66, 159-172, 1997). The cationic surfactant cetyl pyridinuim chloride was utilized to entrap Baker's yeast and Brewer's yeast inside a reverse micelle.

Micelles have also been used as a reaction media. For example, a micelle has been used to study the kinetic and synthetic applications of the dehydrobromination of 2-(p-nitrophenyl)ethyl bromide. Additionally, micelles have found use as an emulsifier for emulsion polymerizations.

Micelles have been utilized for drug delivery. For example, an AB block copolymer has been investigated for the micellar delivery of hydrophobic drugs. Transport and metabolism of thymidine analogues has been investigated via intestinal absorption utilizing a micellar solution of sodium glycocholate. Additionally, several examples of micelle use in transdermal applications have appeared. For example, sucrose laurate has been utilized for topical preparations of cyclosporin A.

Complexation of Nucleic Acids with Polycations

Polymers are used for drug delivery for a variety of therapeutic purposes. Polymers have also been used for the delivery of nucleic acids (polynucleotides and oligonucleotides) to cells for therapeutic purposes that have been termed gene therapy or anti-sense therapy. One of the several methods of nucleic acid delivery to the cells is the use of DNA-polycation complexes. It was shown that cationic proteins like histones and protamines or synthetic polymers like polylysine, polyarginine, polyomithine, DEAE dextran, polybrene, and polyethylenimine were effective intracellular delivery agents while small polycations like spermine were ineffective. Furthermore, polycations are a very convenient linker for attaching specific receptors to DNA and as result, DNA-polycation complexes can be targeted to specific cell types. However, DNA-polycation complexes sometimes interact with each other to form aggregates, or contain multiple DNA molecules in the complex, thereby affecting the size of the complx.

There are a variety of molecules (gene transfer enhancing signals) that can be covalently attached to the gene in order to enable or enhance its cellular transport. These include signals that enhance cellular binding to receptors, cytoplasmic transport to the nucleus and nuclear entry or release from endosomes or other intracellular vesicles.

For Example, nuclear localizing signals can enhance the entry of the gene into the nucleus or can direct the gene into the proximity of the nucleus. Such nuclear transport signals can be a protein or a peptide such as the SV40 large T ag NLS or the nucleoplasmin NLS. Other molecules include ligands that bind to cellular receptors on the membrane surface increasing contact of the gene with the cell. These can include targeting group such as agents that target to the asialoglycoprotein receptor by using asiologlycoproteins or galactose residues. Other proteins such as insulin, EGF, or transferrin can be used for targeting. Peptides that include the RGD sequence can be used to target many cells. Chemical groups that react with sulfhydryl or disulfide groups on cells can also be used to target many types of cells. Folate and other vitamins can also be used for targeting. Other targeting groups include molecules that interact with membranes such as fatty acids, cholesterol, dansyl compounds, and amphotericin derivatives.

The size of a DNA complex may be a factor for gene delivery in vivo. Many times, the size of DNA that is of interest is large, and one method of delivery utilizes compaction techniques. The DNA complex needs to cross the endothelial barrier and reach the parenchymal cells of interest. The largest endothelia fenestrae (holes in the endothelial barrier) occur in the liver and have average diameter of 100 nm. The trans-epithelial pores in other organs are much smaller, for example, muscle endothelium can be described as a structure which has a large number of small pores with a radius of 4 nm, and a very low number of large pores with a radius of 20-30 nm. (Rippe, B. Physiological Rev, 1994). The size of the DNA complex is also important for the cellular uptake process. After binding to the target cells the DNA complex should be taken up by endocytosis. Since the endocytic vesicles have a homogenous internal diameter of about 100 nm in hepatocytes, and are of similar size in other cell types, the DNA is compacted to be smaller than 100 nm.

Compaction (Condensation) of DNA

There are two major approaches for compacting (condensing) DNA:
1. Multivalent cations with a charge of three or higher have been shown to condense DNA. These include spermidine, spermine, Co(NH³)₆³⁺,Fe³⁺, and natural or synthetic polymers such as histone H1, protamine, polylysine, and polyethylenimine. One analysis has shown DNA condensation to be favored when 90% or more of the charges along the sugar-phosphate backbone are neutralized (Wilson R W et al. Biochemistry 18, 2192-2196, 1979).
2. Polymers (neutral or anionic) which can increase repulsion between DNA and its surroundings have been shown to compact DNA. Most significantly, spontaneous DNA self-assembly and aggregation process have been shown to result from the confinement of large amounts of DNA, due to excluded volume effect (Strzelecka T E et al. Biopolymers 30, 57-71, 1990). Since self-assembly is associated with locally or macroscopically crowded DNA solutions, it is expected, that DNA insertion into small water cavities with a size comparable to the DNA will tend to form mono or oligomolecular compact structures.

### SUMMARY OF THE INVENTION

The present invention provides for the delivery of polynucleotides, and biologically active compounds into parenchymal cells within tissues in vitro and in vivo, utilizing reverse micelles. A biologically active compound is a compound having the potential to react with biological components. Pharmaceuticals, proteins, peptides, hormones, cytokines, antigens and nucleic acids are examples of biologically active compounds. The reverse micelle may be negatively-charged, zwitterionic, or neutral. Additionally, the present invention provides a process for the modification of polynucleotides, and biologically active compounds within a reverse micelle.

In a preferred embodiment, a method for the modification of a polynucleotide is described comprising: inserting a nucleic acid into a reverse micelle, and adding a second component that reacts with the polynucleotide to form a modified polynucleotide. The second component can be dissolved in a reverse micelle or dissolved in an appropriate organic solvent.

Additional components may then be added to the modified polynucleotide. The modified polynucleotide can then be isolated by the disruption of the reverse micelle.

In another preferred embodiment, a method for the modification of a polynucleotide is described comprising: inserting a nucleic acid into a reverse micelle, and adding a second component that reacts with a reactive group(s) on the polynucleotide to form a modified polynucleotide. The second component can be dissolved in a reverse micelle or dissolved in an appropriate organic solvent. Additional components may then be added to the modified polynucleotide. The modified polynucleotide can then be isolated by the disruption of the reverse micelle.

In another preferred embodiment, the preparation of a polynucleotide complex is described comprising: inserting a nucleic acid into a reverse micelle, and adding a second component to form a polynucleotide complex. The second component can be dissolved in an organic solvent, or can in a reverse micelle. A third component can then added to the polynucleotide complex that reacts with the second component. For example, a crosslinker that reacts with the second component may be added to the polynucleotide complex. Other components can be added to the polynucleotide complex while in the reverse micelle, such as a delivery enhancing ligand, another polyion, targeting group or another compound. The resulting polynucleotide complex can then be isolated by the disruption of the reverse micelle.

In another preferred embodiment, the preparation of a polynucleotide complex is described comprising: inserting a nucleic acid into a reverse micelle, and adding a second component to form a polynucleotide complex. The second component can be dissolved in an organic solvent, or can in a reverse micelle. A third component is then added to the polynucleotide complex that reacts with the second component. For example, a crosslinker may be added to the polynucleotide complex that reacts with the second component. Other components can be added to the polynucleotide complex while in the reverse micelle, such as a delivery enhancing ligand, another polyion, targeting group or another compound. The resulting polynucleotide complex can then be isolated by the disruption of the reverse micelle, and the polynucleotide complex can be delivered to a cell.

### BRIEF DESCRIPTION OF THE DRAWINGS

FIG. 1. Circular dichroism spectra measured for samples of plasmid DNA added to a mixture of Brij30/TMP or DNA alone at 30° C. The ellipticity value for control samples prepared without DNA were subtracted from the experimental samples.

### DETAILED DESCRIPTION

A process is described for the modification of a polynucleotide or for the preparation of a polynucleotide complex within a reverse micelle. The reverse micelle has the property to compact the nucleic acid, and can be utilized as a medium for constructing the polynucleotide complex. Following complex formation, the reverse micelle can be destroyed and the polynucleotide complex can be isolated. Formation of reverse micelles containing nucleic acid is described in U.S. application Ser. No. 10/627,247, which is incorporated herein by reference.

A process is described for the modification of a polynucleotide or for the preparation of a polynucleotide complex within a reverse micelle. The reverse micelle has the property to compact the nucleic acid, and can be utilized as a medium for constructing the polynucleotide complex. Following complex formation, the reverse micelle can be destroyed and the polynucleotide complex can be isolated.

More specifically, the invention describes the modification of a compacted polynucleotide within a reverse micelle. Traditional methods for polynucleotide compaction generally involve methods that would inhibit a reaction taking place on the compacted polynucleotide. For example, polynucleotides are compacted with polymers that can react with the modification reagent. Additionally, aggregation of the polynucleotides can be problematic.

In the present invention, the polynucleotide is taken up in a reverse micelle, where the polynucleotide is still available for chemical reaction, by adding the polynucleotide in aqueous solution to an organic solution of a surfactant within the range of W0 where reverse micelles are formed. A reagent for modifying the polynucleotide can then be added, either directly to the reverse micelle containing solution or to an organic solution of a surfactant (forming a second reverse micelle solution) and mixing the two reverse micelle solutions. After an appropriate amount of time for the modification to proceed, the reverse micelle can be disrupted or destroyed by adding aqueous and organic solutions to afford a two phase solution. The aqueous layer is then washed with organic solvents to remove organic soluble material, and diluted to an appropriate concentration to afford the modified polynucleotide.

Additionally, the present invention describes the preparation of a polynucleotide complex within a reverse micelle. Formulation and preparation of polynucleotide complexes can involve a number of steps in order to impart different functionality to the complex. Traditionally, these steps must be conducted in aqueous solutions due to the solubility of the polynucleotide. However, some reagents (for example crosslinking reagents and cell targeting signals) beneficial is complex preparation can be unstable or insoluble in aqueous solutions. The present invention provides for the preparation of complexes that might otherwise be problematic since an organic solvent is utilized in which added solubility or stability may be beneficial due to components of the complex. In addition, the invention provides for the disruption of the reverse micelle and the isolation of the complex for delivery to cells.

In the present invention, the polynucleotide is taken up in a reverse micelle, where the polynucleotide is available for complex formation, by adding the polynucleotide in aqueous solution to an organic solution of a surfactant within the range of W0 where reverse micelles are formed. A second component can then be added, either directly to the reverse micelle containing solution or to an organic solution of a surfactant (forming a second reverse micelle solution) and mixing the two reverse micelle solutions. After an appropriate amount of time for the components to mix and a complex to form, additional components can be added. For example a polymer can be added to the polynucleotide in a reverse micelle and mixed, resulting in a polynucleotide-polymer complex. An additional component can then be added, for example a crosslinking agent, in order to crosslink the polymer of the polynucleotide-polymer complex. After an appropriate amount of time for the crosslinking reaction to occur, the reverse micelle can be disrupted or destroyed by adding aqueous and organic solutions to afford a two phase solution. The aqueous layer is then washed with organic solvents to remove organic soluble material, and diluted to an appropriate concentration to afford the polynucleotide complex. Under the present invention, reagents that have little solubility or are hydrolytically active can be utilized in complex formation.

A chemical reaction can take place within the reverse micelle. Compounds capable of reacting with nucleic acid in the environment of the reverse micelle can be used to modify the nucleic acid. Modification of the nucleic acid can be selected from the group comprising: crosslinking, labeling, and attaching a targeting ligand, steric stabilizer, peptide, membrane active compound, or other group that facilitates delivery of the nucleic acid to a cell.

Complexation of the nucleic acid can also occur within the reverse micelle. These complexes can be further modified while the complex is still within the reverse micelle or after disruption of the reverse micelle. Modification can be selected from the group comprising: crosslinking, labeling, and attaching a targeting ligand, steric stabilizer, peptide, membrane active compound, or other group that facilitates delivery of the nucleic acid to a cell.

Disrupting or cleaving the micelle means to separate the solution into a two phase solution.

Complex—Two molecules are combined to form a complex through a process called complexation or complex formation if the are in contact with one another through noncovalent interactions such as electrostatic interactions, hydrogen bonding interactions, or hydrophobic interactions.

Delivery particle—A delivery particle is the polynucleotide complex that is delivered to cells.

Polynucleotide—The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations on DNA, RNA and other natural and synthetic nucleotides.

DNA may be in form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups. An anti-sense polynucleotide is a polynucleotide that interferes with the function of DNA and/or RNA. Antisense polynucleotides include, but are not limited to: morpholinos, 2′-O-methyl polynucleotides, DNA, RNA and the like. SiRNA comprises a double stranded structure typically containing 15-50 base pairs and preferably 21-25 base pairs and having a nucleotide sequence identical or nearly identical to an expressed target gene or RNA within the cell. Interference may result in suppression of expression. The polynucleotide can be a sequence whose presence or expression in a cell alters the expression or function of cellular genes or RNA. In addition, DNA and RNA may be single, double, triple, or quadruple stranded. Double, triple, and quadruple stranded polynucleotide may contain both RNA and DNA or other combinations of natural and/or synthetic nucleic acids.

A delivered polynucleotide can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell. Polynucleotides may contain an expression cassette coded to express a whole or partial protein, or RNA. An expression cassette refers to a natural or recombinantly produced polynucleotide that is capable of expressing a gene(s). The term recombinant as used herein refers to a polynucleotide molecule that is comprised of segments of polynucleotide joined together by means of molecular biological techniques. The cassette contains the coding region of the gene of interest along with any other sequences that affect expression of the gene. A DNA expression cassette typically includes a promoter (allowing transcription initiation), and a sequence encoding one or more proteins. Optionally, the expression cassette may include, but is not limited to, transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include, but is not limited to, translation termination signals, a polyadenosine sequence, internal ribosome entry sites (IRES), and non-coding sequences.

The polynucleotide may contain sequences that do not serve a specific function in the target cell but are used in the generation of the polynucleotide. Such sequences include, but are not limited to, sequences required for replication or selection of the polynucleotide in a host organism.

A polynucleotide can be used to modify the genomic or extrachromosomal DNA sequences. This can be achieved by delivering a polynucleotide that is expressed. Alternatively, the polynucleotide can effect a change in the DNA or RNA sequence of the target cell. This can be achieved by hybridization, multistrand polynucleotide formation, homologous recombination, gene conversion, or other yet to be described mechanisms.

The term gene generally refers to a polynucleotide sequence that comprises coding sequences necessary for the production of a therapeutic polynucleotide (e.g., ribozyme) or a polypeptide or precursor. The polypeptide can be encoded by a full length coding sequence or by any portion of the coding sequence so long as the desired activity or functional properties (e.g., enzymatic activity, ligand binding, signal transduction) of the full-length polypeptide or fragment are retained. The term also encompasses the coding region of a gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term gene encompasses both cDNA and genomic forms of a gene. A genomic form or clone of a gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced out from the nuclear or primary transcript; introns therefore are absent in the messenger RNA (mRNA) transcript. The mRNA functions during translation to specify the sequence or order of amino acids in a nascent polypeptide. The term non-coding sequences also refers to other regions of a genomic form of a gene including, but not limited to, promoters, enhancers, transcription factor binding sites, polyadenylation signals, internal ribosome entry sites, silencers, insulating sequences, matrix attachment regions. These sequences may be present close to the coding region of the gene (within 10,000 nucleotide) or at distant sites (more than 10,000 nucleotides). These non-coding sequences influence the level or rate of transcription and translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, and immunogenicity. One example of covalent modification of nucleic acid involves the action of LabelIT reagents (Mirus Corporation, Madison, Wis.).

As used herein, the term gene expression refers to the process of converting genetic information encoded in a gene into RNA (e.g., mRNA, rRNA, tRNA, or snRNA) through transcription of a deoxyribonucleic gene (e.g., via the enzymatic action of an RNA polymerase), and for protein encoding genes, into protein through translation of mRNA. Gene expression can be regulated at many stages in the process. Up-regulation or activation refers to regulation that increases the production of gene expression products (i.e., RNA or protein), while down-regulation or repression refers to regulation that decrease production. Molecules (e.g., transcription factors) that are involved in up-regulation or down-regulation are often called activators and repressors, respectively.

An RNA function inhibitor comprises any polynucleotide or nucleic acid analog containing a sequence whose presence or expression in a cell causes the degradation of or inhibits the function or translation of a specific cellular RNA, usually an mRNA, in a sequence-specific manner. Inhibition of RNA can thus effectively inhibit expression of a gene from which the RNA is transcribed. RNA function inhibitors are selected from the group comprising: siRNA, interfering RNA or RNAi, dsRNA, RNA Polymerase III transcribed DNAs encoding siRNA or antisense genes, ribozymes, and antisense nucleic acid, which may be RNA, DNA, or artificial nucleic acid. SiRNA comprises a double stranded structure typically containing 15-50 base pairs and preferably 21-25 base pairs and having a nucleotide sequence identical or nearly identical to an expressed target gene or RNA within the cell. Antisense polynucleotides include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA or linear RNAs that can function as antisense RNA. The RNA function inhibitor may be polymerized in vitro, recombinant RNA, contain chimeric sequences, or derivatives of these groups. The RNA function inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded.

Transfection—The process of delivering a polynucleotide to a cell has been commonly termed transfection or the process of transfecting and also it has been termed transformation. The term transfecting as used herein refers to the introduction of a polynucleotide or other biologically active compound into cells. The polynucleotide may be used for research purposes or to produce a change in a cell that can be therapeutic. The delivery of a polynucleotide for therapeutic purposes is commonly called gene therapy. The delivery of a polynucleotide can lead to modification of the genetic material present in the target cell. The term stable transfection or stably transfected generally refers to the introduction and integration of an exogenous polynucleotide into the genome of the transfected cell. The term stable transfectant refers to a cell which has stably integrated the polynucleotide into the genomic DNA. Stable transfection can also be obtained by using episomal vectors that are replicated during the eukaryotic cell division (e.g., plasmid DNA vectors containing a papilloma virus origin of replication, artificial chromosomes). The term transient transfection or transiently transfected refers to the introduction of a polynucleotide into a cell where the polynucleotide does not integrate into the genome of the transfected cell. If the polynucleotide contains an expressible gene, then the expression cassette is subject to the regulatory controls that govern the expression of endogenous genes in the chromosomes. The term transient transfectant refers to a cell which has taken up a polynucleotide but has not integrated the polynucleotide into its genomic DNA.

Intravascular and vessel—The term intravascular refers to an intravascular route of administration that enables a polymer, oligonucleotide, or polynucleotide to be delivered to cells more evenly distributed than direct injections. Intravascular herein means within an internal tubular structure called a vessel that is connected to a tissue or organ within the body of an animal, including mammals. Vessels comprise internal hollow tubular structures connected to a tissue or organ within the body. Bodily fluid flows to or from the body part within the cavity of the tubular structure. Examples of bodily fluid include blood, lymphatic fluid, or bile. Examples of vessels include arteries, arterioles, capillaries, venules, sinusoids, veins, lymphatics, and bile ducts. Afferent blood vessels of organs are defined as vessels which are directed towards the organ or tissue and in which blood flows towards the organ or tissue under normal physiological conditions. Conversely, efferent blood vessels of organs are defined as vessels which are directed away from the organ or tissue and in which blood flows away from the organ or tissue under normal physiological conditions. In the liver, the hepatic vein is an efferent blood vessel since it normally carries blood away from the liver into the inferior vena cava. Also in the liver, the portal vein and hepatic arteries are afferent blood vessels in relation to the liver since they normally carry blood towards the liver. Insertion of the inhibitor or inhibitor complex into a vessel enables the inhibitor to be delivered to parenchymal cells more efficiently and in a more even distribution compared with direct parenchymal injections.

Modification—A molecule is modified, to form a modification through a process called modification, by a second molecule if the two become bonded through a covalent bond. That is, the two molecules form a covalent bond between an atom form one molecule and an atom from the second molecule resulting in the formation of a new single molecule. A chemical covalent bond is an interaction, or bond, between two atoms in which there is a sharing of electron density. Modification also means an interaction between two molecules through a noncovalent bond. For example crown ethers can form noncovalent bonds with certain amine groups.

Salt—A salt is any compound containing ionic bonds; i.e., bonds in which one or more electrons are transferred completely from one atom to another. Salts are ionic compounds that dissociate into cations and anions when dissolved in solution and thus increase the ionic strength of a solution.

Pharmaceutically Acceptable Salt—Pharmaceutically acceptable salt means both acid and base addition salts.

Pharmaceutically Acceptable Acid Addition Salt—A pharmaceutically acceptable acid addition salt is a salt that retains the biological effectiveness and properties of the free base, is not biologically or otherwise undesirable, and is formed with inorganic acids such as hydrochloric acid, hydrobromic acid, sulfuric acid, nitric acid, phosphoric acid and the like, and organic acids such as acetic acid, propionic acid, pyruvic acid, maleic acid, malonic acid, succinic acid, fumaric acid, tartaric acid, citric acid, benzoic acid, mandelic acid, methanesulfonic acid, ethanesulfonic acid, p-toluenesulfonic acid, salicylic acid, trifluoroacetic acid, and the like.

Pharmaceutically Acceptable Base Addition Salt—A pharmaceutically acceptable base addition salt is a salts that retains the biological effectiveness and properties of the free acid, is not biologically or otherwise undesirable, and is prepared from the addition of an inorganic organic base to the free acid. Salts derived from inorganic bases include, but are not limited to, sodium, potassium, calcium, lithium, ammonium, magnesium, zinc, and aluminum salts and the like. Salts derived from organic bases include, but are not limited to, salts of primary secondary, and tertiary amines, such as methylamine, triethylamine, and the like.

Salt Stabilized Complex—A salt stabilized complex is a complex that shows stability when exposed to 150 mM NaCl solution. Stability in this case is indicated by a stable particle size reading (less than a 20% change over 30 min) for the complex in 150 mM NaCl solution. Stability in this case is also indicated by no decondensation of the DNA (less than a 20% change over 30 min) within the complex for the complex in 150 mM NaCl solution.

Interpolyelectrolyte Complexes—An interpolyelectrolyte complex is a noncovalent interaction between polyelectrolytes of opposite charge.

Charge, Polarity, and Sign—The charge, polarity, or sign of a compound refers to whether or not a compound has lost one or more electrons (positive charge, polarity, or sign) or gained one or more electrons (negative charge, polarity, or sign).

Functional group—Functional groups include cell targeting signals, nuclear localization signals, compounds that enhance release of contents from endosomes or other intracellular vesicles (releasing signals), and other compounds that alter the behavior or interactions of the compound or complex to which they are attached.

Cell targeting signals—Cell targeting signals are any signals that enhance the association of the biologically active compound with a cell. These signals can modify a biologically active compound such as drug or nucleic acid and can direct it to a cell location (such as tissue) or location in a cell (such as the nucleus) either in culture or in a whole organism. The signal may increase binding of the compound to the cell surface and/or its association with an intracellular compartment. By modifying the cellular or tissue location of the foreign gene, the function of the biologically active compound can be enhanced. The cell targeting signal can be, but is not limited to, a protein, peptide, lipid, steroid, sugar, carbohydrate, (non-expressing) polynucleic acid or synthetic compound. Cell targeting signals such as ligands enhance cellular binding to receptors. A variety of ligands have been used to target drugs and genes to cells and to specific cellular receptors. The ligand may seek a target within the cell membrane, on the cell membrane or near a cell. Binding of ligands to receptors typically initiates endocytosis. Ligands include agents that target to the asialoglycoprotein receptor by using asiologlycoproteins or galactose residues. Other proteins such as insulin, EGF, or transferrin can be used for targeting. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as lipids, fatty acids, cholesterol, dansyl compounds, and amphotericin derivatives. In addition viral proteins could be used to bind cells. After interaction of a compound or complex with the cell, other targeting groups can be used to increase the delivery of the biologically active compound to certain parts of the cell.

Nuclear localization signals—Nuclear localizing signals enhance the targeting of the pharmaceutical into proximity of the nucleus and/or its entry into the nucleus during interphase of the cell cycle. Such nuclear transport signals can be a protein or a peptide such as the SV40 large T antigen NLS or the nucleoplasmin NLS. These nuclear localizing signals interact with a variety of nuclear transport factors such as the NLS receptor (karyopherin alpha) which then interacts with karyopherin beta. The nuclear transport proteins themselves could also function as NLS's since they are targeted to the nuclear pore and nucleus. For example, karyopherin beta itself could target the DNA to the nuclear pore complex. Several peptides have been derived from the SV40 T antigen. Other NLS peptides have been derived from the hnRNP A1 protein, nucleoplasmin, c-myc, etc. These include a short NLS (H—CGYGPKKKRKVGG-OH, SEQ ID 1) or long NLS's (H—CKKKSSSDDEATADSQHST-PPKKKRKVEDPKDFPSELLS—OH, SEQ ID 2 and H—CKKKWDDEATADSQHSTPPKKK-RKVEDPKDFPSELLS—OH, SEQ ID 3). Other NLS peptides have been derived from M9 protein (CYNDFGNYNNQSSNFGPMKQGNFGGRSSGPY, SEQ ID 4), E1A (H—CKRGPKRPRP—OH, SEQ ID 5), nucleoplasmin (H—CKKAVKRPAATKKAGQAKKKKL-OH, SEQ ID 6),and c-myc (H—CKKKGPAAKRVKLD-OH, SEQ ID 7).

Membrane active compounds—Many biologically active compounds, in particular large and/or charged compounds, are incapable of crossing biological membranes. In order for these compounds to enter cells, the cells must either take them up by endocytosis, i.e., into endosomes, or there must be a disruption of the cellular membrane to allow the compound to cross. In the case of endosomal entry, the endosomal membrane must be disrupted to allow for movement out of the endosome and into the cytoplasm. Either entry pathway into the cell requires a disruption or alteration of the cellular membrane. Compounds that disrupt membranes or promote membrane fusion are called membrane active compounds. These membrane active compounds, or releasing signals, enhance release of endocytosed material from intracellular compartments such as endosomes (early and late), lysosomes, phagosomes, vesicle, endoplasmic reticulum, golgi apparatus, trans golgi network (TGN), and sarcoplasmic reticulum. Release includes movement out of an intracellular compartment into the cytoplasm or into an organelle such as the nucleus. Releasing signals include chemicals such as chloroquine, bafilomycin or Brefeldin A1 and the ER-retaining signal (KDEL sequence), viral components such as influenza virus hemagglutinin subunit HA-2 peptides and other types of amphipathic peptides. The control of when and where the membrane active compound is active is crucial to effective transport. If the membrane active agent is operative in a certain time and place it would facilitate the transport of the biologically active compound across the biological membrane. If the membrane active compound is too active or active at the wrong time, then no transport occurs or transport is associated with cell rupture and cell death. Nature has evolved various strategies to allow for membrane transport of biologically active compounds including membrane fusion and the use of membrane active compounds whose activity is modulated such that activity assists transport without toxicity. Many lipid-based transport formulations rely on membrane fusion and some membrane active peptides' activities are modulated by pH. In particular, viral coat proteins are often pH-sensitive, inactive at neutral or basic pH and active under the acidic conditions found in the endosome.

Cell penetrating compounds—Cell penetrating compounds, which include cationic import peptides (also called peptide translocation domains, membrane translocation peptides, arginine-rich motifs, cell-penetrating peptides, and peptoid molecular transporters) are typically rich in arginine and lysine residues and are capable of crossing biological membranes. In addition, they are capable of transporting molecules to which they are attached across membranes. Examples include TAT (GRKKRRQRRR, SEQ ID 8), VP22 peptide, and an ANTp peptide (RQIKIWFQNRRMKWKK, SEQ ID 9). Cell penetrating compounds are not strictly peptides. Short, non-peptide polymers that are rich in amines or guanidinium groups are also capable of carrying molecules crossing biological membranes. Like membrane active peptides, cationic import peptides are defined by their activity rather than by strict amino acid sequence requirements.

Interaction Modifiers—An interaction modifier changes the way that a molecule interacts with itself or other molecules relative to molecule containing no interaction modifier. The result of this modification is that self-interactions or interactions with other molecules are either increased or decreased. For example cell targeting signals are interaction modifiers which change the interaction between a molecule and a cell or cellular component. Polyethylene glycol is an interaction modifier that decreases interactions between molecules and themselves and with other molecules.

Linkages—An attachment that provides a covalent bond or spacer between two other groups (chemical moieties). The linkage may be electronically neutral, or may bear a positive or negative charge. The chemical moieties can be hydrophilic or hydrophobic. Preferred spacer groups include, but are not limited to C1-C12 alkyl, C1-C12 alkenyl, C1-C12 alkynyl, C6-C18 aralkyl, C6-C18 aralkenyl, C6-C18 aralkynyl, ester, ether, ketone, alcohol, polyol, amide, amine, polyglycol, polyether, polyamine, thiol, thio ether, thioester, phosphorous containing, and heterocyclic. The linkage may or may not contain one or more labile bonds.

Bifunctional—Bifunctional molecules, commonly referred to as crosslinkers, are used to connect two molecules together, i.e. form a linkage between two molecules. Bifunctional molecules can contain homo or heterobifunctionality.

Labile Bond—A labile bond is a covalent bond that is capable of being selectively broken. That is, the labile bond may be broken in the presence of other covalent bonds without the breakage of the other covalent bonds. For example, a disulfide bond is capable of being broken in the presence of thiols without cleavage of other bonds, such as carbon-carbon, carbon-oxygen, carbon-sulfur, carbon-nitrogen bonds, which may also be present in the molecule. Labile also means cleavable.

Labile Linkage—A labile linkage is a chemical compound that contains a labile bond and provides a link or spacer between two other groups. The groups that are linked may be chosen from compounds such as biologically active compounds, membrane active compounds, compounds that inhibit membrane activity, functional reactive groups, monomers, and cell targeting signals. The spacer group may contain chemical moieties chosen from a group that includes alkanes, alkenes, esters, ethers, glycerol, amide, saccharides, polysaccharides, and heteroatoms such as oxygen, sulfur, or nitrogen. The spacer may be electronically neutral, may bear a positive or negative charge, or may bear both positive and negative charges with an overall charge of neutral, positive or negative.

pH-Labile Linkages and Bonds—pH-labile refers to the selective breakage of a covalent bond under acidic conditions (pH<7). That is, the pH-labile bond may be broken under acidic conditions in the presence of other covalent bonds that are not broken.

Amphiphilic and Amphipathic Compounds—Amphipathic, or amphiphilic, compounds have both hydrophilic (water-soluble) and hydrophobic (water-insoluble) parts.

Polymers—A polymer is a molecule built up by repetitive bonding together of smaller units called monomers. In this application the term polymer includes both oligomers which have two to about 80 monomers and polymers having more than 80 monomers. The polymer can be linear, branched network, star, comb, or ladder types of polymer. The polymer can be a homopolymer in which a single monomer is used or can be copolymer in which two or more monomers are used. Types of copolymers include alternating, random, block and graft.

Other Components of the Monomers and Polymers—The polymers have other groups that increase their utility. These groups can be incorporated into monomers prior to polymer formation or attached to the polymer after its formation. These groups include: Targeting Groups—such groups are used for targeting the polymer-nucleic acid complexes to specific cells or tissues. Examples of such targeting agents include agents that target to the asialoglycoprotein receptor by using asialoglycoproteins or galactose residues. Other proteins such as insulin, EGF, or transferrin can be used for targeting. Protein refers to a molecule made up of 2 or more amino acid residues connected one to another as in a polypeptide. The amino acids may be naturally occurring or synthetic. Peptides that include the RGD sequence can be used to target many cells. Chemical groups that react with thiol, sulfhydryl, or disulfide groups on cells can also be used to target many types of cells. Folate and other vitamins can also be used for targeting. Other targeting groups include molecules that interact with membranes such as fatty acids, cholesterol, dansyl compounds, and amphotericin derivatives.

The polymers can also contain cleavable groups within themselves. When attached to the targeting group, cleavage leads to reduce interaction between the complex and the receptor for the targeting group. Cleavable groups include but are not restricted to disulfide bonds, diols, diazo bonds, ester bonds, sulfone bonds, acetals, ketals, enol ethers, enol esters, enamines and imines.

Polyelectrolyte—A polyelectrolyte, or polyion, is a polymer possessing more than one charge, i.e. the polymer contains groups that have either gained or lost one or more electrons. A polycation is a polyelectrolyte possessing net positive charge, for example poly-L-lysine hydrobromide. The polycation can contain monomer units that are charge positive, charge neutral, or charge negative, however, the net charge of the polymer must be positive. A polycation also can mean a non-polymeric molecule that contains two or more positive charges. A polyanion is a polyelectrolyte containing a net negative charge. The polyanion can contain monomer units that are charge negative, charge neutral, or charge positive, however, the net charge on the polymer must be negative. A polyanion can also mean a non-polymeric molecule that contains two or more negative charges. The term polyelectrolyte includes polycation, polyanion, zwitterionic polymers, and neutral polymers. The term zwitterionic refers to the product (salt) of the reaction between an acidic group and a basic group that are part of the same molecule.

Steric Stabilizer—A steric stabilizer is a long chain hydrophilic group that prevents aggregation by sterically hindering particle to particle or polymer to polymer electrostatic interactions. Examples include: alkyl groups, PEG chains, polysaccharides, alkyl amines. Electrostatic interactions are the non-covalent association of two or more substances due to attractive forces between positive and negative charges.

Buffers—Buffers are made from a weak acid or weak base and their salts. Buffer solutions resist changes in pH when additional acid or base is added to the solution.

Biological, Chemical, or Biochemical reactions—Biological, chemical, or biochemical reactions involve the formation or cleavage of ionic and/or covalent bonds.

Reactive—A compound is reactive if it is capable of forming either an ionic or a covalent bond with another compound. The portions of reactive compounds that are capable of forming covalent bonds are referred to as reactive functional groups or reactive groups.

Steroid—A steroid derivative means a sterol, a sterol in which the hydroxyl moiety has been modified (for example, acylated), a steroid hormone, or an analog thereof. The modification can include spacer groups, linkers, or reactive groups.

Sterics—Steric hindrance, or sterics, is the prevention or retardation of a chemical reaction because of neighboring groups on the same molecule.

Lipid—Any of a diverse group of organic compounds that are insoluble in water, but soluble in organic solvents such as chloroform and benzene. Lipids contain both hydrophobic and hydrophilic sections. The term lipids is meant to include complex lipids, simple lipids, and synthetic lipids.

Complex Lipids—Complex lipids are the esters of fatty acids and include glycerides (fats and oils), glycolipids, phospholipids, and waxes.

Simple Lipids—Simple lipids include steroids and terpenes.

Synthetic Lipids—Synthetic lipids includes amides prepared from fatty acids wherein the carboxylic acid has been converted to the amide, synthetic variants of complex lipids in which one or more oxygen atoms has been substituted by another heteroatom (such as Nitrogen or Sulfur), and derivatives of simple lipids in which additional hydrophilic groups have been chemically attached. Synthetic lipids may contain one or more labile groups.

Fats—Fats are glycerol esters of long-chain carboxylic acids. Hydrolysis of fats yields glycerol and a carboxylic acid—a fatty acid. Fatty acids may be saturated or unsaturated (contain one or more double bonds).

Oils—Oils are esters of carboxylic acids or are glycerides of fatty acids.

Glycolipids—Glycolipids are sugar containing lipids. The sugars are typically galactose, glucose or inositol.

Phospholipids—Phospholipids are lipids having both a phosphate group and one or more fatty acids (as esters of the fatty acid). The phosphate group may be bound to one or more additional organic groups.

Wax—Waxes are any of various solid or semisolid substances generally being esters of fatty acids.

Fatty Acids—Fatty acids are considered the hydrolysis product of lipids (fats, waxes, and phosphoglycerides).

Surfactant—A surfactant is a surface active agent, such as a detergent or a lipid, which is added to a liquid to increase its spreading or wetting properties by reducing its surface tension. A surfactant refers to a compound that contains a polar group (hydrophilic) and a non-polar (hydrophobic) group on the same molecule. A cleavable surfactant is a surfactant in which the polar group may be separated from the nonpolar group by the breakage or cleavage of a chemical bond located between the two groups, or to a surfactant in which the polar or non-polar group or both may be chemically modified such that the detergent properties of the surfactant are destroyed.

Detergent—Detergents are compounds that are soluble in water and cause nonpolar substances to go into solution in water. Detergents have both hydrophobic and hydrophilic groups

Micelle—Micelles are microscopic vesicles that contain amphipathic molecules but do not contain an aqueous volume that is entirely enclosed by a membrane. In micelles the hydrophilic part of the amphipathic compound is on the outside (on the surface of the vesicle). In inverse micelles the hydrophobic part of the amphipathic compound is on the outside. The inverse micelles thus contain a polar core that can solubilize both water and macromolecules within the inverse micelle.

Liposome—Liposomes are microscopic vesicles that contain amphipathic molecules and contain an aqueous volume that is entirely enclosed by a membrane.

Microemulsions—Microemulsions are isotropic, thermodynamically stable solutions in which substantial amounts of two immiscible liquids (water and oil) are brought into a single phase due to a surfactant or mixture of surfactants. The spontaneously formed colloidal particles are globular droplets of the minor solvent, surrounded by a monolayer of surfactant molecules. The spontaneous curvature, H0 of the surfactant monolayer at the oil/water interface dictates the phase behavior and microstructure of the vesicle. Hydrophilic surfactants produce oil in water (O/W) microemulsions (H0>0), whereas lipophilic surfactants produce water in oil (W/O) microemulsions.

Hydrophobic Groups—Hydrophobic groups indicate in qualitative terms that the chemical moiety is water-avoiding. Typically, such chemical groups are not water soluble, and tend not to form hydrogen bonds.

Hydrophilic Groups—Hydrophilic groups indicate in qualitative terms that the chemical moiety is water-preferring. Typically, such chemical groups are water soluble, and are hydrogen bond donors or acceptors with water.

Substructure—Substructure means the chemical structure of the compound and any compounds derived from that chemical structure from the replacement of one or more hydrogen atoms by any other atom or change in oxidation state. For example if the substructure is succinic anhydride, then methylsuccinic anhydride, 2,2-dimethylsuccinic anhydride, 3-oxabicyclo[3.1.0]hexane-2,4-dione, maleic anhydride, citriconic anhydride, and 2,3-dimethylmaleic anhydride have the same substructure.

### EXAMPLES

### Example 1

### Synthesis of 5,5′-Dithiobis[succinimidyl(2-nitrobenzoate)]

5,5′-dithiobis(2-nitrobenzoic acid) (50.0 mg, 0.126 mmol) and N-hyroxysuccinimide (29.0 mg, 0.252 mmol) were taken up in 1.0 mL dichloromethane. Dicylohexylcarbodiimide (52.0 mg, 0.252 mmol) was adhded and the reaction mixture was stirred overnight at room temperature. After 16 hr, the reaction mixture was partitioned in EtOAc/H₂O. The organic layer was washed 2× with H₂O, 1× with brine, dried (with MgSO₄) and concentrated under reduced pressure. The residue was taken up in CH₂Cl₂, filtered, and purified by flash column chromatography on silica gel (130×30 mm, EtOAc:CH₂Cl₂ 1:9 eluent) to afford 42 mg (56%) 5,5′-dithiobis[succinimidyl(2-nitrobenzoate)] (EdiNHS) as a white solid. H¹ NMR (DMSO) ∂7.81-7.77 (d, 2H), 7.57-7.26 (m, 4H), 3.69 (s, 8 H).

### Example 2

### General Preparation of Peptides

Peptides were prepaired by standard solid phase peptide synthesis using an ABI433A Peptide Synthesizer (Applied Biosystems), employing FastMoc chemistry. Peptides were sysnthesized on the 0.1 or 1.0 mmol scale. Deprotections and cleavage of the resin were accomplished utilizing standard deprotection techniques. Peptides were purified by reverse phase HPLC to at least a 90% purity level, and verified by mass spectroscopy (Sciex API 150EX). Peptide A: Peptide MC1089, Sequence: H₂N-GIGAILKVLATGLPTLISWIKNKRKQ-OH (SEQ ID 10).

### Example 3

### pCILuc DNA/Labeled Poly-L-Lysine Interaction

To poly-L-lysine (PLL) (4 mg, Sigma Chemical Company) in potassium phosphate buffer (pH 8, 0.1 mL) was added 7-Chloro-4-nitrobenz-2-oxa-1,3-diazole (NBD-Cl) (0.4 mg, Sigma Chemical Company). The solution was heated at 37° C. for 2 h, cooled, and purified by gel-filtration on Sephadex G-25. The fluorescence was determined (Hitachi, model F-3010, excitation wavelength=466 nm, emission wavelength=540 nm), and the level of modification was estimated to be 5%. To the NBD-PLL (5 μg) in HEPES (25 mM, pH 7.8) and EDTA (0.5 mM) (1 mL), was added varying amounts of pDNA, and the fluorescence was again determined.

| | pDNA (μg) | | | | |
|---|---|---|---|---|---|
| | 0 | 1 | 2 | 4 | 6 |
| Fluorescence | 41 | 27 | 21 | 17 | 16 |
| Intensity of | | | | | |
| NBD | | | | | |

These results indicate that compaction (or condensation) of a fluorescently labeled polyion (in this example PLL) by a polyion of opposite charge (in this example DNA) results in a decrease in fluorescence intensity (quantum yield of fluorescence) of the fluorephore.

### Example 4

### pCILuc DNA/Polycation Interaction in a Reverse Micelle

NBD-PLL was mixed with Polyoxyethylene(4) lauryl ether (Brij 30) in 2,2,4-trimethylpentane (TMP) (1:7.3 v/v) to form a reverse micelle containing PLL. This reverse micelle solution was then mixed with an equal volume of reverse micelle containing solution formed from of Brij 30/TMP (1:7.3 v/v) that contained either HEPES (25 mM, pH 7.8) and EDTA (0.5 mM) without or with various amounts of pDNA (various amounts). After 10 min at ambient temperature, the fluorescence was determined for each sample.

| Conditions | I₅₄₀ |
|---|---|
| 0.5 mL TMP with 5 μg NBD-PLL in 20 μL buffer + | 87 |
| 0.5 mL TMP with 20 μL buffer | |
| 0.5 mL TMP with 5 μg NBD-PLL in 20 μL buffer + | 64 |
| 0.5 mL TMP with 3.7 μg | |
| DNA in 20 μL buffer | |
| 0.5 mL TMP with 5 μg NBD-PLL in 20 μL buffer + | 38 |
| 0.5 mL TMP with 11.1 μg | |
| DNA20 μL buffer | |

The decreased fluorescence of the NBD-PLL indicated interaction of the DNA with the PLL therefore indicating that pDNA in reverse micelles can interact with PLL in reverse micelles.

### Example 5

### pCILuc DNA/Crosslinked Polycation Interaction

To a solution of pDNA (35 μg) in HEPES (25 mM, pH 7.8), EDTA (0.5 mM), and NaCl (100 mM) (24 μL) was added Polyoxyethylene(4) lauryl ether (Brij 30) (Aldrich Chemical Company)/2,2,4-trimethylpentane (TMP) (Aldrich Chemical Company) (510 μL, 1:7.3 v/v). Poly-L-lysine (PLL) (95 μg, Sigma Chemical Company) in HEPES (25 mM, pH 7.8), EDTA (0.5 mM), and NaCl (100 mM) (12 μL) was added to Brij 30/TMP (290 μL, 1:7.3 v/v). The resulting solutions were mixed and heated to 40° C. for 30 min at which time dimethyl 3,3′-dithiobispropionimidate-2HCl (DTBP, Pierce Chemical Company) in DMSO (various amounts of a 29.5 mg/mL solution) were added. The solution was heated to 40° C. for 25 min at which time HEPES (25 mM, pH 7.8), EDTA (0.5 mM), and NaCl (100 mM) (200 μL) was added, followed by EtOH (50 μL) and EtOAc (0.5 mL) to disrupt the reverse micelles. After mixing and centrifugation, the aqueous layer was washed with EtOAc (2×1 mL) and Ether (2×1 mL). The samples were spun (5 min, 12000 rpm) and dialyzed for 16 h against HEPES (25 mM, pH 7.8) and NaCl (100 mM) to recover the DNA. The UV absorption was determined (Perkin Elmer UV/VIS Spectrophotometer, Model Lambda 6). A solution of TOTO6 (Zeng, Z., Clark, S. M., Mathies, R. A., Glazer, A. N. Analytical Biochemistry, 252, 110-114, 1997) (2 μL, 0.5 mg/mL in water) was added and the fluorescence was determined (Hitchi, Model F-3010, excitation wavelength=509 nm, emission wavelength=540 nm).

| | Amount of DTBP | | |
|---|---|---|---|
| Number | (μL) | % DNA Recovery | Fluorescence |
| 35 μg DNA | — | 100 | 120.4 |
| (no treatment) | | | |
| 1 | 0 | 3 | 0.275 |
| 2 | 3 | 14 | 1.76 |
| 3 | 6 | 19 | 3.07 |
| 4 | 12 | 24 | 4.02 |

The results indicate that the pDNA-PLL complex can be partly extracted from reverse micelles after the PLL has been crosslinked with DTBP. The pDNA in the extracted complexes is compacted because it does not interact with the fluorescent intercalator TO6.

### Example 6

### pCILuc DNA1 Polyethylenimine Complexes in Reverse Micelles

pDNA was modified to a level of approximately 1 rhodamine per 100 bases using Mirus LABEL-IT® Rhodamine kit (Rhodamine Containing DNA Labeling Reagent, Mirus Bio Corporation). Labeled pDNA (14 μg) was taken up in HEPES (25 mM, pH 7.8) and EDTA (0.5 mM) (various amounts) and added to Polyoxyethylene(4) lauryl ether (Brij 30)/2,2,4-trimethylpentane (TMP) (1 mL, 1:7.3 v/v). The fluorescence and turbidity of each sample was determined. Polyethylenimine (PEI) (30 μg, Sigma Chemical Company) in HEPES (25 mM, pH 7.8) and EDTA (0.5 mM) (3 μL) was added to each sample. After 30 min the florescence and turbidity of each sample was determined.

| | |
|---|---|
| No PEI Added | With PEI Added |

| Sample W0 | I₆₁₀ | Turbidity | I₆₁₀ | Turbidity |
|---|---|---|---|---|
| DNA alone | 28.45 | 31 | 8.7 | 76 |
| in buffer | | | | |
| 0.67 | 14.8 | 105 | 11.5 | 164 |
| 1.51 | 9.7 | 103 | 10.2 | 144 |
| 2.35 | 11.0 | 85 | 11.8 | 114 |
| 4.03 | 18.3 | 105 | 15.9 | 137 |
| 5.71 | 26.0 | 182 | 18.0 | 217 |
| 9.06 | 31.6 | 4200 | 17.8 | 4734 |

W0 = molar ratio of water to surfactant

The decrease in fluorescence indicates that a polycation can be added to DNA in reverse micelles and the polycation can interact with the DNA.

### Example 7

### Oxidation Within a Reverse Micelle

Cysteine LABEL-IT® was prepared by amidation of amino LABEL-IT® (Mirus Bio Corporation Madison Wis.) with N-Boc-S-trityl cysteine (Sigma Chemical Company) utilizing dicyclohexylcarbodiimide (Aldrich Chemical Co.) as the coupling agent. The product was purified by precipitation with diethyl ether. The trityl and Boc protecting groups were removed with trifluoroacetic acid. The resulting free thiol group was protected with Aldrithiol-2® (Aldrich Chemical Co.) as the pyridyldithio mixed disulfide and was purified by diethyl ether precipitation and confirmed by mass spectrometry (Sciex API 150EX).

pCILuc DNA (pDNA) was modified with Cysteine LABEL-IT® at weight ratios of 0.1:1 and 0.2:1 (reagent:DNA) at 37° C. for 1 h. The labeled DNA was purified by ethanol precipitation. The purified DNA was reconstituted in 20 mM MOPS pH 7.5, 0.1 mM EDTA buffer at a final concentration of 1 μg/μL. The level of PDP-cysteine reagent incorporation on DNA was estimated from the optical adsorption ratio of pyridine-2-thione (λₘₐₓ 343 nm and extinction coefficient E=8.08×10³) and DNA (λₘₐₓ 260 nm and extinction E=6.6×10³) after treatment of 15 μg of the modified DNA with 5 mM dithiothreitol (Sigma Chemical Co.) for 1.5 h at 20° C.

The labeled DNA was treated with 20 mM dithiothreitol (DTT, Sigma Chemical Co.) for 1 h at 4° C. to generate free thiols on the labeled plasmid. Reverse micelles were prepared by dissolving 82 μL of 1 μg/μL Cys-DNA in 2.2 mL C₁₂E₄/TMP (W0=6.58). The mixtures were agitated using a vortex stirrer until a transparent solution was obtained (usually about 2 min). After formation of the micelles, sodium periodate was added to a final concentration of 2 mM with respect to the total aqueous portion to oxidize the thiols to disulfides. The samples were centrifuged for 1 min at 14,000 rpm to remove any aggregates. A control reaction was prepared following the same procedure using non-labeled DNA. The samples were incubated at 4° C. for 2 h. The reverse micelle system was disrupted with the addition of 55 μL ethanol, 275 μL of 20 mM MOPS pH 7.5, 0.1 mM EDTA buffer, and 1.1 mL ethyl acetate. The reaction was vortexed and separated into two layers via centrifugation. The aqueous layer was washed twice with 2 mL ethyl acetate and once with 3 mL diethyl ether. The samples were then analyzed by agarose gel electrophoresis.

Agarose gel electrophoresis, indicated that periodate oxidized, cysteine DNA was found to remain in the well (indicating intramolecular oxidation of cysteine groups (formation of disulfide bonds) on the DNA). The non-oxidized cysteine DNA migrated into the gel similarly to the unmodified DNA control.

### Example 8

### Mouse Tail Vein Injections of Oxidized Cysteine-pDNA(pCI Luc) Complexes Formed in a Reverse Micelle

pCILuc DNA (pDNA) was modified with Cysteine LABEL-IT® at weight ratios of 0.1:1 and 0.2:1 (reagent:DNA) at 37° C. for 1 h. The labeled DNA was treated with 20 mM dithiothreitol (DTT, Sigma Chemical Co.) for 1 h at 4° C. to generate free thiols on the labeled plasmid. Reverse micelles were prepared as described in Example 7. For each weight ratio, both an oxidized (sodium periodate added to the reverse micelle) and a non-oxidized sample (no sodium periodate was added) were prepared. The pDNA was isolated as previously described.

Five complexes were prepared as follows:
Complex I: pDNA (pCI Luc, 30 μg) in 7.5 mL Ringers.
Complex II: 0.1:1 cysteine labeled pDNA (pCI Luc, 30 μg) non-oxidized, in 7.5 mL Ringers.
Complex III: 0.1:1 cysteine labeled pDNA (pCI Luc, 300 μg) oxidized in the reverse micelle,in 7.5 mL Ringers.
Complex IV: 0.2:1 cysteine labeled pDNA (pCI Luc, 30 μg) non-oxidized, in 7.5 mL Ringers.
Complex V: 0.2:1 cysteine labeled pDNA (pCI Luc, 30 μg) oxidized in the reverse micelle, in 7.5 mL Ringers.

Hydrodynamic tail vein injection was performed on ICR mice (n=3) to delivery the plasmid DNA to liver cells. Tail vein injections of 2.5 mL of the complex were preformed using a 30 gauge, 0.5 inch needle. One day after injection, the animal was sacrificed, and a luciferase assay was conducted on the liver. Luciferase expression was determined as previously reported (Wolff J A et al. 1990). A Lumat L B 9507 (EG&G Berthold, Bad-Wildbad, Germany) luminometer was used.

| Complex | RLU |
|---|---|
| Complex I | 17,113,000 RLU |
| Complex II | 21,111,000 RLU |
| Complex III | 11,998,000 RLU |
| Complex IV | 2,498,000 RLU |
| Complex V | 4,498,000 RLU |

The luciferase assay indicates that the pDNA that is oxidized within the reverse micelle is functional and able to be expressed.

### Example 9

### Conducting a Chemical Modification of pDNA in a Reverse Micelle—Labeling pDNA with a Cy3 Fluorophore

pMir48 (4 μL of 2.5/mg/ml 5 mM Hepes, pH7.9, 0.1 mM EDTA) was added to 0.7 mL of Brij 30/TMP (1:7.3 v/v) and mixed until a clear solution (W0=1.00). Cy3-LABEL-IT® (various amounts in DMSO) was added to the DNA in reverse micelles and the solution was mixed for 1 h. After 1 h, the micelles were disrupted by adding 50 μL EtOH, 200 μL 5 mM Hepes, pH7.9, and 2 mL EtOAc. Following centrifugation, the aqueous layer was washed 2× with 2 mL EtOAc and 2× with 2 mL Et₂O. 20 μL 5 M NaCl was added followed by 5 mL EtOH. The samples were placed at −20° C. for 1 h. The samples were spun down and the resulting pellet was washed 2× with 2 mL 70% EtOH. Pelletes were dissolved in 1 ml 5 mM Hepes, pH7.9.

The amount of pDNA recovered in the reactions was determined from the absorbance at λ₂₆₀ on a DU530 Life Science UV/Vis Spectrophotometer (Beckman). The amount of CY®3 present was determined from the absorbance at λ₄₄₉. Fluorescence intensity was determined on a Cary Eclipse Fluorescence Spectrophotometer (Varian Inc.), with λₑₓ=549, λₑₘ=570.

| Sample | | | | | |
|---|---|---|---|---|---|
| pDNA:LABEL-IT ® | | μg Cy3 | | Fl./ | |
| (wt:wt), | μg | per μg | Flourescence | pmol | Fl./ |
| LABEL-IT ® (μg/μl) | pDNA | DNA | Intensity. | Cy3 | A260 |
| Inverse micelle | 8.3 | 8.0 | 54.52 | 0.818 | 5452 |
| (1:0.5), 1 | | | | | |
| Inverse micelle | 8.7 | 22.2 | 103.0 | 0.533 | 3550 |
| (1:1), 50 | | | | | |
| Inverse micelle | 4.75 | 43.5 | 101.1 | 0.489 | 3261 |
| (1:1), 50 | | | | | |
| Inverse micelle | 5.0 | 101 | 213.5 | 0.421 | 2809 |
| (1:5), 50 | | | | | |

The results show that the condensed pDNA can be covalently modified within a reverse micelle.

### Example 10

### Preparation of Polycation from the Imidate of N,N-Dimethylformamide (MC1015)

Method A: A solution of HCl in diethyl ether (1 mL, 1.0 M, Aldrich Chemical Company) was cooled to −78° C. in a dry ice/acetone bath under N₂. N,N-Dimethylformamide (85 mg, 1.2 mmol, anhydrous) was added dropwise. The resulting precipitate was isolated by centrifugation, washed with diethylether (2×2 mL), dried under a N₂ stream, and placed under high vacuum to afford the imidate (30 mg, 23% yield). The resulting imidate was dissolved in DMF (300 μL, anhydrous) and the resulting solution was allowed to stand at room temperature for 3 days. The resulting product is the polycation MC1015.

Method B: To a solution of HCl in diethyl ether (20.0 mL, 1.0 M) was added anhydrous N,N-Dimethylformamide (1.55 mL, 20 mmol) dropwise, resulting in a slightly yellow precipitate. An additional 20 mL diethyl ether was added and the resulting suspension was mixed. The ether was decanted and the precipitate was washed with diethyl ether (3×40 mL), dried under a stream of N₂, and placed under high vacuum to afford 1.22 g (56% yield) of the imidate as a slightly yellow solid. To the imidate was added anhydrous DMF, and the resulting solution was heated to reflux under N₂. The solution was cooled and the polymer precipitated with diethyl ether. The precipitate was washed with diethyl ether (5×5 mL), and dried under vacuum to afford 635 mg of yellow rust solid.

Method C: N,N-Dimethylformamide (47.2 g, 0.646 mol, anhydrous) was cooled to −20° C., and HCl gas was bubbled through the solution over 30 min. The resulting solution was warmed to room temperature under a blanket of N₂ to afford a clear viscous solution. After 3 days at room temperature the solution contains the polycation MC1015.

Elemental Analysis of MC1015 indicates:

| Element | Wt % |
|---|---|
| C | 27.04 |
| H | 9.86 |
| O | 21.24 |
| N | 16.41 |
| Cl | 25.45 |

### Example 11

### Preparation of a pDNA Complex within a Reverse Micelle and Isolation of the Complex

The following pMir48 complexes were prepared.

Complex I. pMir48 (50 μg in 20 μL 5 mM Hepes, pH7.9, 0.1 mM EDTA)/MC1015 (14.5 μL 86 mg/mL DMF)/MC1089 peptide (1 eq, 8.54 μL 10 mg/mL DMSO)/EdNHS (1 eq, 8.9 μL 10 mg/mL DMSO)/Galactose amine (10 eq, 16.3 μL 20 mg/mL DMSO)—Micellar Formulation—Brij30/TMP (1:7.3 v/v). To 1 mL of Brij 30/TMP (1:7.3 v/v) was added pMir 48 (50 μg in 20 μL 5 mM Hepes, pH7.9, 0.1 mM EDTA) and the solution was mixed until clear (Micelles, W0=3.49). MC1015 was added and the solution mixed for 30 min. MC1089 was added and the solution was mixed for 30 min. EdiNHS was added and the solution was again mixed for 30 min. Galactose amine was added and the solution was mixed for 30 min. To disrupt the micelles, 150 μL of EtOH was added followed by 850 μL isotonic glucose, then 10 mL EtOAc. Following centrifugation, the aqueous layer was washed with 1×10 mL EtOAc and 1×10 mL Et₂O. Isotonic glucose was added to 1 mL final volume.

Complex II. pMir48 (50 μg in 20 μL 5 mM Hepes, pH7.9, 0.1 mM EDTA)/500 μL isotonic glucose/MC1015 (14.5 μL 86 mg/mL DMF)/MC1089 peptide (1 eq, 8.54 μL 10 mg/mL DMSO)/EdNHS (1 eq, 8.9 μL 10 mg/mL DMSO)/galactose amine (10 eq, 16.3 μL 20 mg/mL DMSO)/431.8 μL isotonic glucose. Final volume=1 mL isotonic glucose−Non-Micellar Formulation

Complex III. DNA/PLL/MC1089 peptide/EdiNHS (⅓ wt/1 chg/1.5 mol eq, Brij30 Micelle). pMir48 (4 μL of 2.5×5 mM Hepes, pH7.9, 0.1 m EDTA) was added to 0.7 mL of Brij 30/TMP (1:7.3 v/v) and mixed until a clear solution (W0=1.00). To this micellar solution was added PLL (3.0 μL 10 mg/mL in DMSO, 3 wt eq) and the solution was mixed for 30 min. MC1089 (1.7 μL 10 mg/mL DMSO, 1 chg eq) was added and the solution was mixed for 30 min. EdiNHS (1.8 μL 10 mg/mL DMSO, 1 mol eq) was added and the solution was again mixed for 30 min.

To disrupt the micelles, 55 μL of EtOH was added followed by 250 μL OPTI-MEM®, then 1.5 mL EtOAc. Following centrifugation, the aqueous layer was washed 1×1.5 mL EtOAc and 1×1.5 mL Et₂O. OPTI-MEM® was added to 0.5 mL final volume.

Complex IV. DNA/PLL/MC1089 peptide/EdiNHS (⅓ wt/1 chg/1.5 mol eq). pMir48 (4 μL of 2.5×5 mM Hepes, pH7.9, 0. 1 mM EDTA) was added to 0.5 mL of isotonic glucose and mixed. PLL (3.0 μL 10 mg/mL in DMSO, 3 wt eq) was added and the solution was mixed for 30 min. MC1089 (1.7 μL 10 mg/mL DMSO, 1 chg eq) was added and the solution was mixed for 30 min. EdiNHS (1.8 μL 10 mg/mL DMSO, 1 mol eq) was added and the solution was again mixed for 30 min.

Complexes were analyzed for particles by dynamic light scattering (Zeta Plus Particle Sizer, Brookhaven Instrument Corporation, λ=532). Poly acrylic acid (pAcAc, 15 μL of 100 mg/mL solution in water) was added to each sample and the particle size was again determined. DTT (15 μL of 1 M solution in water) was added to each sample and the particle size was again determined.

Results:

| | | Particle size (counts) | Particle size (counts) |
|---|---|---|---|
| Complex | Particle size (counts) | after pAcAc | after DTT |
| Complex I | 147 nm (1095 kcps) | 92 nm (735 kcps) | 9048 nm (500 kcps) |
| Complex II | 292 nm (1585 kcps) | 5.5 nm (388 kcps) | 4.9 nm (330 kcps) |
| Complex III | 157 nm (2695 kcps) | 132 nm (1863 kcps) | 3.2 nm (490 kcps) |
| Complex IV | 9990 nm (2860 kcps) | — | — |

Particle sizing on complex I indicates 147 nm particles that are stable to polyanion challenge. Upon cleaving the crosslinker with DTT, the particle is not stable to the polyanion. Particle sizing on complex II indicates larger 292 nm particles that are not stable to polyanion challenge. The 5.5 nm particles do not contain pDNA indicating the crosslinking in solution was not efficient. Similar results were obtained with PLL complexes indicating that the EdiNHS crosslinking is more efficient when utilized in a reverse micelle.

### Example 12

### Hepa Cell Transfection

Samples were prepared as follows:

Complex I. DNA/MC1089 peptide/EdiNHS (⅓ chg/1.5 mol eq, Solution formulation) pMir48 (4 μL of 2.5×5 mM Hepes, pH7.9, 0.1 mM EDTA) was added to 500 μL of isotonic glucose. MC1089 (5.1 μL 10 mg/mL DMSO, 3 chg eq) was added and the solution was mixed for 30 min. EdiNHS (2.7 μL 10 mg/mL DMSO, 1.5 mol eq) was added and the solution was again mixed for 30 min. Diluted with OPTI-MEMS to 1 μg/100 μL final concentration.

Complex II. DNA/MC1089 peptide/EdiNHS (⅓ chg/1.5 mol eq, Micellar formulation). pMir48 (4 μL of 2.5×5 mM Hepes, pH7.9, 0.1 mM EDTA) was added to 0.7 mL of Brij 30/TMP (1:7.3 v/v) and mixed until a clear solution (W0=1.00). To this micellar solution was added MC1089 (5.1 μL 10 mg/mL DMSO, 3 chg eq) and the solution was mixed for 30 min. EdiNHS (2.7 μL 10 mg/mLDMSO, 1.5 mol eq) was added and the solution was again mixed for 30 min. To disrupt the micelles, 55 μL of EtOH was added followed by 250 μL OPTI-MEM®, then 1.5 mL EtOAc. Following centrifugation, the aqueous layer was washed 1×1.5 mL EtOAc and 1×1.5 mL Et₂O. Added OPTI-MEM® to 0.5 mL final volume, and diluted further for 1 μg DNA in 100 μL OPTI-MEM® samples.

Complex III. DNA/MC1089 peptide/EdiNHS (⅓ chg/1.5 mol eq, Micellar formulation). pMir48 (4 μL of 2.5×5 mM Hepes, pH7.9, 0.1 mM EDTA) was added to 0.7 mL of Brij 30/TMP (1:7.3 v/v) and mixed until a clear solution (W0=1.00). To this micellar solution was added MC1089 (5.1 μL 10 mg/mL DMSO, 3 chg eq) and the solution was mixed for 30 min. EdiNHS (2.7 μL 10 mg/mL DMSO, 1.5 mol eq) was added and the solution was again mixed for 30 min. To disrupt the micelles, 55 μL of EtOH was added followed by 250 μL OPTI-MEM®, then 1.5 mL EtOAc. Following centrifugation, the aqueous layer was washed 1×1.5 mL EtOAc and 1×1.5 mL Et₂O. Added OPTI-MEM(& to 0.5 mL final volume.

Complex IV. DNA/PLL/MC1089 peptide/EdiNHS (⅓ wt/1 chg/1.5 mol eq). pMir48 (4 μL of 2.5×5 mM Hepes, pH7.9, 0.1 mM EDTA) was added to 0.5 mL of isotonic glucose and mixed. PLL (3.0 μL 10 mg/mL in DMSO, 3 wt eq) was added and the solution was mixed for 30 min. MC1089 (1.7 μL 10 mg/mL DMSO, 1 chg eq) was added and the solution was mixed for 30 min. EdiNHS (1.8 μL 10 mg/mL DMSO, 1 mol eq) was added and the solution was again mixed for 30 min. Diluted with OPTI-MEM® to 1 μg/100 μL final concentration.

Complex V. DNA/PLL/MC1089 peptide/EdiNHS (⅓ wt/1 chg/1.5 mol eq, Brij30 Micelle). pMir48 (4 μL of 2.5×5 mM Hepes, pH7.9, 0.1 mM EDTA) was added to 0.7 mL of Brij 30/TMP (1:7.3 v/v) and mixed until a clear solution (W0=1.00). To this micellar solution was added PLL (3.0 μL 10 mg/mL in DMSO, 3 wt eq) and the solution was mixed for 30 min. MC1089 (1.7 μL 10 mg/mL DMSO, 1 chg eq) was added and the solution was mixed for 30 min. EdiNHS (1.8 μL 10 mg/mL DMSO, 1 mol eq) was added and the solution was again mixed for 30 min.

To disrupt the micelles, 55 μL of EtOH was added followed by 250 μL OPTI-MEM®, then 1.5 mL EtOAc. Following centrifugation, the aqueous layer was washed 1×1.5 mL EtOAc and 1×1.5 mL Et₂O. OPTI-MEM® was added to 0.5 mL final volume.

Hepa cells were maintained in DMEM. Approximately 24 h prior to transfection, cells were plated at an appropriate density in 12-well plates and incubated overnight. Cultures were maintained in a humidified atmosphere containing 5% CO2 at 37° C. The cells were transfected at a starting confluency of 50% by combining 100 μL sample (1-2 μg pDNA per well) with the cells in 1 mL of media. Cells were harvested after 48 h and assayed for luciferase activity using a Lumat LB 9507 (EG&G Berthold, Bad-Wildbad, Germany) luminometer. The amount of luciferase expression was recorded in relative light units. Numbers are the average for two separate wells.

### Hepa Cell Transfection Results

| Complex | RLU Mean |
|---|---|
| I | 635 |
| II | 36,600 |
| III | 573,515 |
| IV | 19,790 |
| V | 17,635 |

Results indicate that pDNA MC1089 peptide complexes prepared in a reverse micelle were better in the transfection compared to a corresponding complex prepared in isotonic glucose. PLL complexes either prepared in a reverse micelle or in isotonic glucose gave similar transfection levels.

### Example 13

### Synthesis off β-D-Glucopyranosyl Dodecane Disulfide

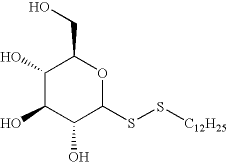

To a solution of dodecane thiol (1.00 mL, 4.17 mmol, Aldrich Chemical Company) in 20 mL CHCl₃ was added sulfuryl chloride (0.74 mL, 9.18 mmol), and the resulting mixture was stirred at room temperature for 18 h. Removal of solvent (aspirator), afforded dodecansulfenyl chloride that was determined to be sufficiently pure by ¹H NMR.

To a solution of dodecansulfenyl chloride (213 mg, 0.899 mmol) in 2.7 mL acetonitrile was added 1-thio-β-D-glucose sodium salt hydrate (200 mg, 0.917 mmol) and 15-crown-5 (0.18 mL, 0.899 mmol, Aldrich Chemical Company). The resulting mixture was stirred at ambient temperature for 3 h, and the solvent removed (aspirator). The residue was triturated with CHCl₃ and filtered. The residue was purified by flash column chromatography on silica gel (0-5% MeOH in CH₂Cl₂). Crystallization (EtOAc) afforded 85 mg (24%) of β-D-glucopyranosyl dodecane disulfide as a fine white solid.

### Experiment 14

### Synthesis of β-D-Glucopyranosyl Decane Disulfide and O-Glycine-β-D-Glucopyranosyl Decane Disulfide

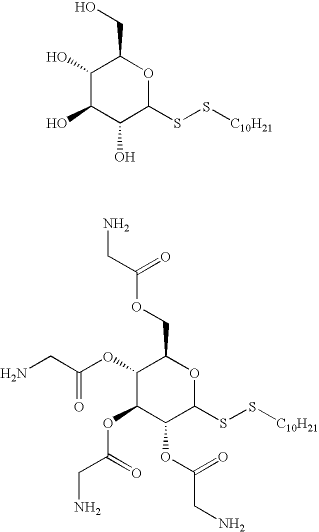

To a solution of decane thiol (0.59 mL, 2.9 mmol) in 11 mL CHCl₃ was added sulfuryl chloride (0.46 mL, 5.7 mmol), and the resulting mixture was stirred at room temperature for 18 h. Removal of solvent (aspirator), afforded decansulfenyl chloride.

To a solution of decansulfenyl chloride (190 mg, 0.92 mmol) in 4 mL acetonitrile was added 1-thio-β-D-glucose sodium salt hydrate (200 mg, 0.92 mmol, Aldrich Chemical Company) and 15-crown-5 (0.18 mL, 0.899 mmol, Aldrich Chemical Company). The resulting mixture was stirred at ambient temperature for 16 h, filtered, and precipitated in Et₂O. The residue was triturated with Et₂O and purified by reverse phase HPLC on an Aquasil C18 column (Keystone Scientific Inc.), 10-90% B, 20 min (A=0.1% TFA in H₂O, B=0.1% TFA in Acetonitrile). Lyophilization afforded 10 mg (3%) of β-D-glucopyranosyl decane disulfide as a fine white solid.

To a solution of β-D-glucopyranosyl decane disulfide (8 mg, 0.02 mmol) in 80 μL THF was added N-Boc glycine (15 mg, 0.09 mmol, Sigma Chemical Company), DCC (18 mg, 0.09 mmol), and a catalytic amount of dimethylaminopyridine. The resulting solution was stirred at ambient temperature for 12 h, and centrifuged to remove the solid. The resulting solution was concentrated under reduced pressure, resuspended in dichloromethane, filtered through a plug of silica gel, and concentrated (aspirator). The Boc protecting group was removed by taking the residue up in 200 μL of 2.5% TIS/50% TFA/dichloromethane for 12 h. Removal of solvent (aspirator), followed by purification by reverse phase HPLC on a Aquasil C18 column (Keystone Scientific Inc.), 10-90% B, 20 min (A=0.1% TFA in H₂0, B=0.1% TFA in Acetonitrile) afforded 0.7 mg (5%) of O-glycine-β-D-glucopyranosyl decane disulfide as a fine white solid following lyophilization.

### Example 15

### Synthesis of β-D-Glucopyranosyl Cholesterol Disulfide

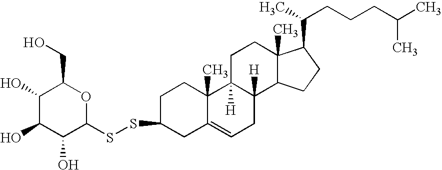

By similar methodology as described in example 14, β-D-glucopyranosyl cholesterol disulfide was isolated (12% yield).

### Experiment 16

### Synthesis of Two Tailed β-D-Glucopyranosyl Disulfide Derivatives. β-D-Glucopyranosyl N-Dodecanoyl-Cysteine-Dodecanoate Disulfide and O-Glycine-β-D-Glucopyranosyl N-Dodecanoyl-Cysteine-Dodecanoate Disulfide

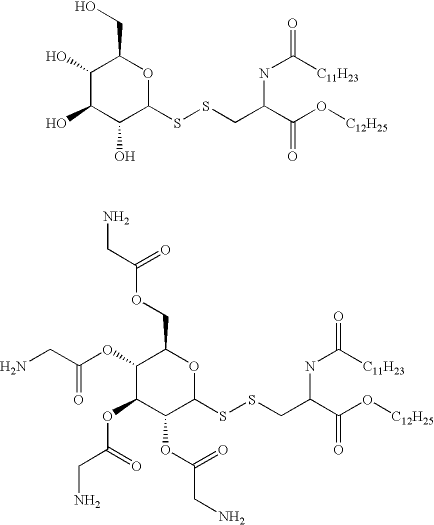

To a solution of N-FMOC-S-Trt-Cysteine (585 mg, 1.0 mmol, NovaBioChem) in 4 mL dichloromethane was added 1-dodecanol (240 mg, 1.3 mmol), DCC (260 mg, 1.3 mmol), and a catalytic amount of dimethylaminopyridine. The resulting solution was stirred at ambient temperature for 30 min, filtered, and purified by flash chromatography on silica gel (10-20% EtOAc/hexane eluent). Removal of solvent (aspirator) afforded 572 mg (76%) of the protected cysteine-dodecanoate.

To a solution of protected cysteine-dodecanoate (572 mg, 0.76 mmol) was added 3 mL of 20% piperidine in DMF. The resulting solution was stirred at ambient temperature for 1 h, and partitioned in EtOAc/H₂O. The aqueous layer was extracted 2×EtOAc. The combined organic layer was washed 2×1N HCl, dried (Na₂SO₄), and concentrated to afford S-Trt-cysteine-dodecanoate. The residue was suspended in 2 mL dichloromethane, and cooled to −20° C. Diisopropylethylamine (0.16 mL, 0.92 mmol) was added followed dodecanoyl chloride (0.26 mL, 1.1 mmol), and the solution was allowed to slowly warm to ambient temperature. After 1 h, the solvent was removed (aspirator), and the residue partitioned in EtOAc/H₂O. The organic layer was washed 2×1 N HCl, 1×brine, dried (Na₂SO₄), and the solvent was removed (aspirator). The resulting residue was suspended in 2% TIS/50% TFA/ dichloromethane to remove the trityl protecting group. After 4 h the solution was concentrated, and the resulting residue was purified by flash column chromatography on silica gel (10-20% EtOAc/hexanes eluent) to afford 180 mg (42%) N-dodecanoyl-cysteine-dodecanoate (M+1=472.6).

To a solution of N-dodecanoyl-cysteine-dodecanoate (180 mg, 0.38 mmol) in 0.5 mL chloroform was added sulfuryl chloride (62 μL, 0.76 mmol). The resulting solution was stirred at ambient temperature for 2 h and the solvent was removed (aspirator). The resulting residue was suspended in 1 mL acetonitrile, and 1-thio-o-D-glucose sodium salt hydrate (85 mg, 0.39 mmol) and 15-crown-5 (76 μL, 0.38 mmol) were added. After 1 h at ambient temperature the solvent was removed (aspirator) and the residue was partitioned in EtOAc/H₂O. The organic layer was concentrated and the resulting residue was purified by flash column chromatography on silica gel (5-10% MeOH/0.1% TFA/dichloromethane eluent) to afford 19 mg (8%) β-D-glucopyranosyl N-dodecanoyl-cysteine-dodecanoate disulfide.

To a solution of β-D-glucopyranosyl N-dodecanoyl-cysteine-dodecanoate disulfide (3.9 mg, 0.0045 mmol) in 100 μL dichloromethane was added N-Boc glycine (3.2 mg, 0.018 mmol), DCC (3.8 mg, 0.018 mmol), and a catalytic amount of dimethylamino-pyridine. The resulting solution was stirred at ambient temperature for 4 h, and filtered. The Boc protecting group was removed by taking the residue up in 2 mL of 1% TIS/50% TFA/dichloromethane for 2 h. Removal of solvent (aspirator), followed by purification by reverse phase HPLC on a Diphenyl column (Vydaq), 20-90% B, 20 min (A=0.1% TFA in H₂O, B=0.1% TFA in Acetonitrile) afforded 3.6 mg (90%) of O-glycine-β-D-glucopyranosyl decane disulfide as a fine white solid following lyophilization.

### Experiment 17

### Synthesis of Disulfide Containing Surfactants

### 1) Synthesis of the Disulfide of Decanethiol and 3-Dimethylamino-Thiopropionamide

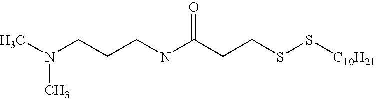

To a solution of thiopropionic acid (0.41 mL, 4.7 mmol) in 18 mL CH₂Cl₂ was added diisopropylethylamine (0.82 mL, 4.7 mmol) followed by trityl chloride (1.4 g, 4.9 mmol). The resulting mixture was stirred at room temperature for 18 h. Removal of solvent (aspirator) afforded a white crystalline solid. The material was partitioned in EtOAc/H₂O, and washed with 0.1 M NaHCO₃ and 1×brine. Concentrated to afford S-trityl thiopropionic acid.

To a solution of S-trityl-thiopropionic acid (0.30 g, 0.86 mmol) in 3.5 mL CH₂Cl₂ was added PyBOP (0.45 g, 0.86 mmol, NovaBioChem). The mixture was stirred at ambient temperature for 5 min and then dimethylaminopropylamine (0.11 mL, 0.86 mmol, Aldrich Chemical Company) was added. The solution was stirred at room temperature for 18 h, and concentrated. The residue was brought up in EtOAc and partitioned in H₂O. The organic layer was washed 2×H₂O, 1×brine, dried (Na₂SO₄), and the solvent removed (aspirator). The resulting residue was suspended in 2% TIS/50% TFA/CH₂Cl₂ (3 mL) to remove the trityl protecting group. After 2 h the solution was concentrated to afford 3-dimethylamino-thiopropionamide.

To a solution of 3-dimethylamino-thiopropionamide (0.082 g, 0.43 mmol) in 1.5 mL dichloromethane was added decanethiolchloride (0.090 g, 0.43 mmol, prepared as in example 15). The resulting solution was stirred at ambient temperature for 20 min. The solvent was removed and the resulting residue was purified by flash column chromatography on silica gel (15% MeOH/CH₂Cl₂ eluent) to afford 17.2 mg (9%) of the disulfide of decanethiol and 3-dimethylamino-thiopropionamide (M+1=363.4).

### 2) Synthesis of the Disulfide of Dodecanethiol and 3-Dimethylamino-Thiopropionamide

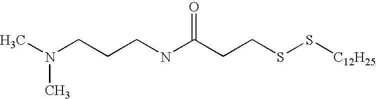

By a similar procedure as above, thiopropyl-dimethylaminopropylamine (0.10 g, 0.52 mmol) in 2.0 mL dichloromethane was added dodecanethiolchloride (0.12 g, 0.52 mmol). The resulting solution was stirred at ambient temperature for 20 min. The solvent was removed and a portion of the resulting residue (160 mg) was purified by flash column chromatography on silica gel (10% MeOH/CH₂Cl₂ eluent) to afford 22.4 mg (14%) of the disulfide of dodecanethiol and 3-dimethylamino-thiopropionamide (M+1=391.4).

### 3) Synthesis of the Disulfide of Decanethiol and Thiopopionic-3-Dimethylaminopropanoate

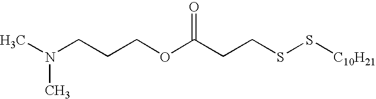

To a solution of trityl-S-thiopropionic acid (0.36 g, 1.0 mmol) in 4.0 mL CH₂Cl₂ was added PyBOP (0.54 g, 1.0 mmol, NovaBioChem). The mixture was stirred at ambient temperature for 5 min before the addition of dimethylaminopropanol (0.12 mL, 1.0 mmol, Aldrich Chemical Company). The solution was stirred at room temperature for 18 h, and concentrated. The residue was brought up in EtOAc and partitioned in H₂O. The organic layer was washed 2×H₂O, 1×brine, dried (Na₂SO₄), and the solvent removed (aspirator). The resulting residue was suspended in 2% TIS/50% TFA/CH₂Cl₂ (3 mL) to remove the trityl protecting group. After 2 h the solution was concentrated to afford thiopopionic-3-dimethylaminopropanoate.

To a solution of thiopopionic-3-dimethylaminopropanoate (0.10 g, 0.52 mmol) in 2 mL dichloromethane was added decanethiolchloride (0.11 g, 0.52 mmol). The resulting solution was stirred at ambient temperature for 20 min. The solvent was removed and a portion of the resulting residue (25 mg) was purified by plug filtration on silica gel (10% MeOH/CH₂Cl₂ eluent) to afford 20.9 mg (84%) of the disulfide of decanethiol and thiopopionic-3-dimethylaminopropanoate (M+1=364.4).

### 4) Synthesis of the Disulfide of Dodecanethiol and Thiopopionic-3-Dimethylaminopropanoate

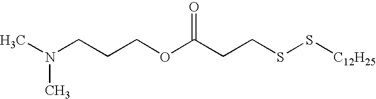

To a solution of thiopopionic-3-dimethylaminopropanoate (0.10 g, 0.52 mmol) in 2 ml dichloromethane was added dodecanethiolchloride (0.11 g, 0.52 mmol). The resulting solution was stirred at ambient temperature for 20 min. The solvent was removed and a portion of the resulting residue (150 mg) was purified by flash column chromatography on silica gel (1% TFA/10% MeOH/CH₂Cl₂ eluent) to afford 38 mg (25%) of the disulfide of decanethiol and thiopopionic-3-dimethylaminopropanoate (N+1=392.4).

### Experiment 18

### Synthesis of Silicone Containing Amphipathic Molecules

### 1) Synthesis of 3-dimethylamino-dimethyloctadecyl silyl ether

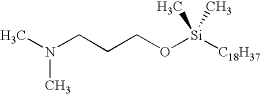

To a solution of 3-dimethylamino-1-propanol (0.873 mmol) in 2 mL chloroform was added dimethyloctadecyl chlorosilane (378 mg, 1.09 mmol) and imidazole (74.2 mg, 1.09 mmol). After 16 hrs at ambient temperature, the solution was partitioned in EtOAc/H₂O with 10% sodium bicarbinate. The organic layer was washed with water, and brine. The solvent was removed (aspirator) to afford 328 mg (91%) of 3-dimethylamino-dimethyloctadecyl silyl ether as a cream colored solid.

### 2) Synthesis of 3-(dimethylamino)-1,2-dimethyloctadecyl silyl ether

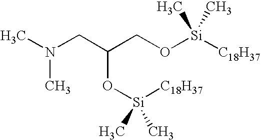

To a solution of 3-(dimethylamino)-1,2-propanediol (50.0 mg, 0.419 mmol, Aldrich Chemical Company) in 2 mL chloroform was added dimethyloctadecyl chlorosilane (328 mg, 0.944 mmol, Aldrich Chemical Company) and imidazole (68.1 mg, 0.944 mmol, Aldrich Chemical Company). After 16 hrs at ambient temperature, the solution was partitioned in EtOAc/H₂O with 10% sodium bicarbinate. The organic layer was washed with water, and brine. The solvent was removed (aspirator) to afford 266 mg (86%) of 3-(dimethylamino)-1,2-dimethyloctadecyl silyl ether as a white solid.

### Example 19

### Demonstration of Micelle Formation with β-D-Glucopyranosyl Dodecane Disulfide, and Micelle Destruction with Dithiothreitol

To a solution of β-D-Glucopyranosyl dodecane disulfide (10 mg) in 1 mL CDCl₃ was added 1 mL H₂O. The sample was rapidly mixed resulting in a thick white emulsion. After 18 h, the organic and aqueous layers were emulsified to approximately 95%. After 4 d, the organic and aqueous layers remained emulsified to approximately 70%. To a 1 mL portion of the emulsion was added 60 μg of dithiothreitol, and the solution was mixed. After 30 min, the emulsion had cleared. 5,5′-Dithiobis(2-nitrobenzioc acid) (1 mg) was added, resulting in a yellow solution, verifying the presence of free sulfide. Analysis also indicated the presence of dodecane thiol and 1-thio-β-D-glucose by TLC.

### Example 20

### Solubilization of pCILuc DNA in Reversed Micelles

pCILuc DNA (pDNA) (11 μg) was taken up in a solution (3-67 μL) of HEPES (25 mM, pH 7.8) and EDTA (0.5 mM). Polyoxyethylene(4) lauryl ether (Brij 30) (1.2 mL) was taken up in 2,2,4-trimethylpentane (TMP) (8.8 ml). To the Brij 30/TMP solution (0.7 mL) was added the pDNA in buffer (3-67 μL). The mixtures were shaken (2 min) resulting in clear solutions. After 10 min the turbidity was determined utilizing a fluorescence spectrophotometer (Hitachi, model F3010, extinction/emission wavelength of 529 nm). W0 is defined as the molar ratio of water to surfactant.

| H₂O (μL) | W0 | Turbidity (529 nm) |
|---|---|---|
| 0 | 0 | 19 |
| 3 | 0.72 | 49 |
| 7 | 1.68 | 63 |
| 12 | 2.87 | 63 |
| 17 | 4.07 | 82 |
| 27 | 6.46 | 2764 |
| 47 | 11.25 | 1565 |
| 67 | 16.04 | 214 |

W0 is defined as the molar ratio of water to surfactant. As the volume of the core aqueous pool increases in the reverse micelle, the aqueous environment begins to match the physical and chemical characteristics of bulk water. The resulting inverse micelle can be referred to as a microemulsion of water in oil. As the amount of water is further increased, a two phase system eventually results. Since W0 is a molar ration, the desired W0 can be achieved by adjusting the amount of water utilized and/or adjusting the amount of surfactant utilized in the complex preparation. Temperature can also effect the structure at a given W0.

At 20° C., the turbidity study indicates that the pDNA solution when added to the Brij 30/TMP results in the formation of reverse micelles. Upon increasing the water content, a two phase system is obtained (W0=6.46), and finally a lamellar phase is obtained (W0=11.25). For a solution of Brij 30 in dodecane the hydrophile-lipophile balance (HLB) temperature has been determined to be approximately 29.2° C. with w/o microemulsion are present for a W0 of less then 10 (Kunieda, H. Langmuir 7,1915, 1991).

### Example 21

### Determination of the Size of PCILuc DNA Contained in Inversed Micelles

Part A. Centrifugation. pCILuc DNA (pDNA) (36 μg) was taken up in a solution of HEPES (25 mM, pH 7.8) and EDTA (0.5 mM) (10 μL, 20 μL, 30 μL, and 50 μL). The resulting solutions were added to a mixture of Polyoxyethylene(4) lauryl ether (Brij 30)/2,2,4-trimethylpentane (TMP) (Aldrich Chemical Company) (1 mL, 1:7.3 v/v) and agitated. The UV adsorption was determined (Perkin Elmer, UV/VIS Spectrophotometer, model Lambda 6) against 10 μL of HEPES (25 mM, pH 7.8) and EDTA (0.5 mM) buffer in Brij 30/TMP (1 mL, 1:7.3 v/v). The samples were centrifuged 5 min at 15000 rpm and the UV adsorption was again determined.

| | | A₂₆₀ before | A₂₆₀ after |
|---|---|---|---|
| Conditions | W0 | centrifugation | centrifugation |
| DNA in buffer | — | 1.07 | 1.07 |
| 10 μL | 1.68 | 1.07 | 1.11 |
| 20 μL | 3.36 | 0.99 | 1.14 |
| 30 μL | 5.04 | 0.97 | 1.01 |
| 50 μL | 8.39 | 2.44 | ND^{a} |

^{a}UV absorption not determined. Solution was two-phase.

At 20° C., micelles that contain pDNA (up to W0 of about 5) are small enough to stay in solution in the course of centrifugation. For these solutions, no change in the UV absorption spectra was recorded as compaired to the UV absorption of pDNA in HEPES (25 mM, pH 7.8) and EDTA (0.5 mM).

Part B. Particle Size of Micelles Without PCILuc DNA. A solution (5-50 μL) of HEPES (25 mM, pH 7.8) and EDTA (0.5 mM) was added to a mixture of Brij 30/TMP (1 mL, 1:7.3 v/v) and agitated (2 min). The samples were centrifuged (1 min) at 12000 rpm and the size of micelles measured (Particle Sizer, Brookhaven Instrument Corporation).

| Volume of buffer (μL) | W0 | Size (nm) |
|---|---|---|
| 0 | 0 | 1.3 |
| 5 | 0.84 | 2.9 |
| 10 | 1.68 | 3.4 |
| 20 | 3.35 | 5.1 |
| 30 | 5.04 | 9.7 |
| 50 | 8.39 | indefinite |

The size of the micelles changes proportionally as the water content increases, from 1.3 nm for “dry” micelles to 9.7 nm for micelles with W0 of about 5. At a higher water content, a two-phase system is present.

Part C. Particle Size of Micelles Containing PCILuc DNA. A solution pDNA in HEPES (25 mM, pH 7.8) and EDTA (0.5 mM) was added to a mixture of Brij 30/TMP (1 mL, 1:7.3 v/v) and agitated (2 min) to form micelles with a W0 of 3.35. The samples were centrifuged (1 min) at 12000 rpm and the size of micelles was measured (Particle Sizer, Brookhaven Instrument Corporation).

| DNA (ng) | Small Micelles (nm) | Large Micelles (nm) |
|---|---|---|
| 0 | 5.1 | — |
| 40 | 4.0 | 16.2 |
| 80 | 4.7 | 48.7 |
| 120 | 4.7 | 62.8 |
| 160 | 4.4 | 51.7 |

Two types of micelles appear to be present in the samples. There are small, “empty” micelles, and large pDNA containing micelles. It appears that the size of micelles containing pDNA increases as the concentration of pDNA increases. The micelle appears to be saturated at a size of 50-60 nm.

### Example 22

### Conformation of PCILuc DNA in Inverse Micelles

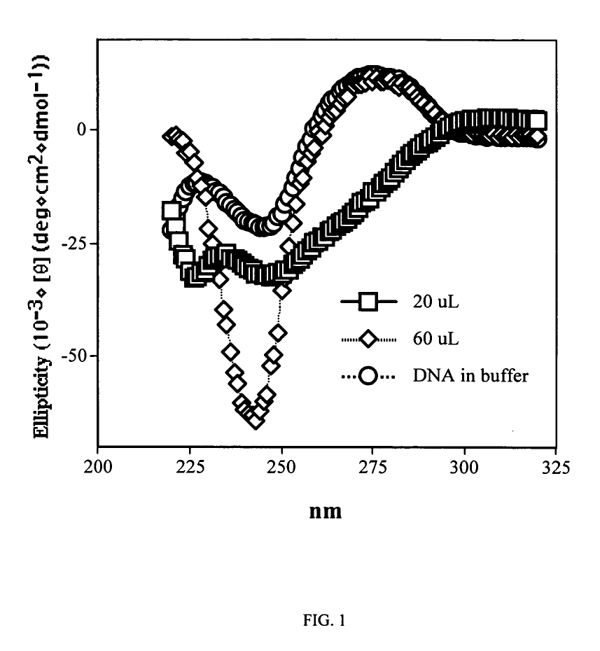

pDNA (60 μg) was taken up in 10 mM potassium phosphate buffer at pH 7.5 (20 μL and 60 μL). The pDNA solutions were added to a mixture of Brij 30/TMP (1 mL, 1:7.3 v/v) and agitated (2 min). The circular dichroism spectra were measured for each sample (cell length=0.5 cm, Spectropolarimeter 62DS, Avive Associates) at 30° C. against control samples prepared without the pDNA (FIG. 1, the ellipticity value for the control samples were subtracted from the experimental samples).

There are shifts in the position of both the positive and negative bands and in the position of the cross-over point for the 20 μL pDNA solution (W0=3.35). Spectra that are similarly shifted are broadly defined as -spectra, and are attributed to a condensed form of pDNA. In contrast the spectra of the 60 μL pDNA solution (W0=10.05) resembles the spectra of DNA in buffer alone in respect to cross-over point. However this spectra is characterized by an increase in the intensity of the negative band (maximum at 240 nm).

### Example 23

### PCILuc DNA Condensation

Part A. Ethidium Bromide. A solution of pDNA in HEPES (25 mM, pH 7.8) and EDTA (0.5 mM) (3-67 μL) containing ethidium bromide (0.9 μg, Sigma Chemical Company) was added to a mixture of Brij 30/TMP (0.7 mL, 1:7.3 v/v) and agitated. After 4 h at ambient temperature, the samples were assayed utilizing a fluorescence spectrophotometer (Hitachi, Model F-3010), with an excitation wavelength of 525 nm and an emission wavelength of 595 nm.

| Volume (μL) | W0 | I/Imax * 100 |
|---|---|---|
| 3 | 0.72 | 15 |
| 7 | 1.68 | 13 |
| 12 | 2.87 | 12 |
| 17 | 4.07 | 12.5 |
| 27 | 6.46 | 23 |
| 47 | 11.25 | 35 |
| 67 | 16.04 | 51 |

The pDNA in reverse micelles of up to about W0=4 is condensed. Additionally, some level of condensation is shown for micelles up to about W0=16.

Part B: Determination of Rhodamine Labeled DNA Condensation in a Reverse Micelle. pDNA was modified to a level of 1 Rhodamine per 100 bases using Mirus' Label It® Rhodamine kit (Rhodamine Containing DNA Labeling Reagent, Mirus Corporation). The modified pDNA (2.5 μg) was solubilized in different volumes of HEPES (25 mM, pH 7.8) and EDTA (0.5 mM) and added to a solution of Brij 30/TMP (0.7 mL, 1:7.3 v/v), and agitated. The fluorescence was determined using a fluorescence spectrophotometer (Hitachi, Model F-3010), at an excitation wavelength of 591 nm, and an emission wavelength of 610 nm.

| Buffer Volume (μL) | W0 | (I₆₁₀ sample/I₆₁₀DNA in buffer) * 100 |
|---|---|---|
| 2 | 0.48 | 104 |
| 4 | 0.96 | 80 |
| 5 | 1.2 | 34 |
| 10 | 2.39 | 31 |
| 12 | 2.87 | 24 |
| 15 | 3.59 | 33 |
| 22 | 5.26 | 32 |
| 32 | 7.66 | 65 |
| 42 | 10 | 106 |
| 52 | 12.45 | 93 |
| 62 | 14.84 | 78 |

It should be noted that around W0=10 turbidity has significant contribution in fluorescence. The assay indicates that under low Water conditions, pDNA does not appear to be condensed. As the amount of water in the system is increased, the fluorescence results indicate that pDNA is condensed within the w/o microemulsion.

### Example 24

### pDNA Condensation in Reverse Micelles

pDNA was modified to a level of 1 Rhodamine per 100 bases using standard procedures (LABEL-IT®). Labeled pDNA (various amounts) was taken up in HEPES (25 mM, pH 7.8) EDTA (0.5 mM) (various amounts) and was mixed with unmodified pDNA (various amounts) to afford 2.5 μg total of pDNA. The resulting solution was added to Brij 30/TMP (0.7 mL, 1:7.3 v/v) and the fluorescence was determined using a fluorescence spectrophotometer (Hitachi, Model F-3010), at an excitation wavelength of 591 nm, and an emission wavelength of 610 nm. For comparison, the fluorescence was also determined for the similar ratios of Rh-labeled pDNA/pDNA containing 2 mM spermidine (Sigma Chemical Company) in HEPES (25 mM, pH 7.8) and EDTA (0.5 mM) (0.7 mL).

| | % of Fluorescence quenching | | | |
|---|---|---|---|---|
| | | | | 2 mM |
| % Rh-DNA | w0 = 2.39 | W0 = 3.59 | W0 = 7.18 | Spermidine |
| 100 | 68.8 | 61.2 | 41.3 | 69.8 |
| 76 | 65.9 | 57.5 | 33.1 | 61 |
| 51 | 59 | 52.2 | 30 | 48 |
| 26 | 55.5 | 50.4 | 28.3 | 26.1 |

The fluorescence data indicates a relatively weak affect of Rh-labeled pDNA dilution by unlabeled pDNA. On the other hand, in the samples containing spermidine, a strong effect of the Rh-pDNA dilution by unlabeled DNA is shown. In reverse micelles, the pDNA condensation starts from monomolecular condensation and therefore show little effect by the dilution protocol. However, in the spermidine containing systems (non-micellular) the strong effect indicates that condensation is multimolecular.

### Example 25

### Transmission Electron Microscope Assay

A drop of Poly-L-lysine (PLL) (30-70 kDa) in water (concentration of 10 mg/mL) was placed on a covered EM grid. The solution was removed, and the grid was dried. A drop of 2,2,4-trimethylpentane (TMP) in various amounts of HEPES (25 mM, pH 7.8) and EDTA (0.5 mM) both with and without PCILuc DNA (pDNA) (7 μg/mL TMP) was placed on the grid. After 5 min, the solution was removed and the grid was washed with TMP (3×) and water (1×), and then stained with Uranyl Acetate.

Samples containing 20 or 60 μL of HEPES (25 mM, pH 7.8) and EDTA (0.5 mM) in TMP (1 mL) failed to show any structures. A sample containing pDNA (7 μg) in HEPES (25 mM, pH 7.8) and EDTA (0.5 mM) in TMP (1 mL) also failed to show any structures. A sample containing pDNA in HEPES (25 mM, pH 7.8) and EDTA (0.5 mM) (20 μL) and TMP (1 mL) demonstrated ring like structures with an external diameter of 59.8±12.5 nm and an internal diameter of 32.9±12.1 nm. A sample of pDNA in HEPES (25 mM, pH 7.8) and EDTA (0.5 mM) (60 μL) and TMP (1 mL) demonstrated long threads with a 7-12 nm diameter. The volume of the terroid ring V=(^{˜}2/4)(Rₒᵤₜ−Rᵢₙ)²(Rₒᵤₜ+Rᵢₙ) equal 41*10³ nm³. The volume of “dry” PCILuc DNA is 6.4*10³ nm³. With consideration of packing parameter every toroid therefore contains five pDNA's.

### Experiment 26

### Application of Reverse Micellar Formulations to Mouse Dermis

Five Complexes were prepared:

Complex I. Doxorubicine hydrochloride was dissolved in water to a final concentration of 5.8 mg/mL. To a solution of 12 μL Brij 30 (Sigma Chemical Company) in 88 μL of tetramethylpentane was added 5 μL of the doxorubicine hydrochloride solution. The sample was vortexed for 2 min resulting in a clear red solution.

Complex II. Doxorubicine hydrochloride was dissolved in water to a final concentration of 50 mg/mL. To a solution of 10 μL of Brij 30 (Sigma Chemical Company) and 2 mg β-D-glucopyranosyl decane disulfide in 190 μL of tetramethylpentane was added 5 μL of the doxorubicine hydrochloride solution. The sample was vortexed for 2 min resulting in a clear red solution.

Complex III. Doxorubicine hydrochloride was dissolved in water to a final concentration of 50 mg/mL. To a solution of 10 μL of Brij 30 (Sigma Chemical Company) and 0.5 mg O-Glycine-β-D-glucopyranosyl decane disulfide in 190 μL of tetramethylpentane was added 5 μL of the doxorubicine hydrochloride solution. The sample was vortexed for 2 min resulting in a clear red solution.

Complex IV. Doxorubicine hydrochloride was dissolved in water to a final concentration of 50 mg/mL. To a solution of 10 μL of Brij 30 (Sigma Chemical Company) and 6 mg 3-dimethylamino-dimethyloctadecyl silyl ether in 190 μL of tetramethylpentane was added 5 μL of the doxorubicine hydrochloride solution. The sample was vortexed for 2 min resulting in a clear red solution.

Complex V. Doxorubicine hydrochloride was dissolved in water to a final concentration of 50 mg/mL. To 200 μL H₂O was added 5 μL of the doxorubicine hydrochloride solution.

ICR mice were anesthetized, and the hair removed from the back of the neck, and on one animal the abdominal skin. After 1 h the animals were sacrificed, and the skin samples removed and examined. The complexes were applied to the dermis as follows:

Complex I. The complex was applied by immersing a cotton swap in the solution, and swabbing the abdominal skin and the dehaired skin on the back of the neck.

Complex II-V. The complex was applied by dropping 50 μL of solution onto the back of the neck.

Fluorescent examination of the skin samples (O.C.T. frozen, UV light). Samples from the application of Complex I were showed a much lower level of positive cells than from Complexes II-IV.

| Com- | | |
|---|---|---|
| plex | Number | Location of the label |
| I | Abdominal | Positive label is restricted to nuclei only with |
| | skin | majority of them being epithelium cells. Small |
| | | portion of positive sells are connective tissue cells |
| | | adjoining to the labeled epithelium cells. |
| | Skin from | Similar pattern of labeling. |
| | the back | |
| II | 7477 | Whole epithelium compartment is very bright, not |
| | | specifically nuclei. Some connective tissue cells in |
| | | deeper part of derma are positive. No positive |
| | | follicular cells. |
| | 7479 | Whole epithelium compartment is very bright, not |
| | | specifically nuclei. Some connective tissue cells in |
| | | deeper part of derma are positive. Very rare positive |
| | | follicular cells. |
| III | 6939 | Whole epithelium compartment is very bright, not |
| | | specifically nuclei. Some follicular cells are |
| | | positive. |
| | 7459 | Whole epithelium compartment is very bright, not |
| | | specifically nuclei. Some follicular cells are positive |
| IV | 7476 | Whole epithelium compartment is positive but less |
| | | than in previous two groups, some connective tissue |
| | | cells in deeper part of derma are positive. |
| | 7460 | Whole epithelium compartment is positive, some |
| | | connective tissue cells in deeper part of derma are |
| | | positive. |
| V | 7474 | Mostly only the skin surface is positive, |
| | | occasionally some deeper cells, probably damaged |
| | | areas (shaving) Cells and nuclei are negative. |
| | 7463 | Mostly only the skin surface is positive, |
| | | occasionally. Cells and nuclei are negative. |

Reverse micelles are able to incorporate doxorubicine hydrochloride and deliver the drug to the epithelium.

The foregoing examples are considered as illustrative only of the principles of the invention. Further, since numerous modifications and changes will readily occur to those skilled in the art, it is not desired to limit the invention to the exact construction and operation shown and described. Therefore, all suitable modifications and equivalents fall within the scope of the invention.

We claim:

1. A process for modifying a nucleic acid comprising:
a) forming a reverse micelle containing the nucleic acid;
b) adding a nucleic acid modifying agent to the nucleic acid in the reverse micelle;
c) disrupting the reverse micelle; and,
d) recovering the modified nucleic acid.

2. The process of claim 1 wherein the nucleic acid contains a reactive group.

3. The process of claim 2 wherein the reactive group consists of a cysteine.

4. A process for condensing a nucleic acid comprising:
a) forming a reverse micelle containing the nucleic acid;
b) adding a polycation to the nucleic acid in the reverse micelle to form a condensed nucleic acid-polycation complex;
c) disrupting the reverse micelle; and,
d) recovering the nucleic acid-polycation complex.

5. The process of claim 4 further comprising adding a modifying agent to the nucleic acid-polycation complex in the reverse micelle.

6. The process of claim 5 wherein the modifying agent consists of a crosslinker.


SEQUENCE LISTING (10 sequences)

SEQ ID NO: 1 - protein, 13 aa, Simian virus 40
CGYGPKKKRKVGG

SEQ ID NO: 2 - protein, 39 aa, Simian virus 40
CKKKSSSDDEATADSQHSTPPKKKRKVEDPKDFPSELLS

SEQ ID NO: 3 - protein, 37 aa, Simian virus 40
CKKKWDDEATADSQHSTPPKKKRKVEDPKDFPSELLS

SEQ ID NO: 4 - protein, 31 aa, Homo sapiens
CYNDFGNYNNQSSNFGPMKQGNFGGRSSGPY

SEQ ID NO: 5 - protein, 10 aa, Human adenovirus type 5
CKRGPKRPRP

SEQ ID NO: 6 - protein, 22 aa, Xenopus laevis
CKKAVKRPAATKKAGQAKKKKL

SEQ ID NO: 7 - protein, 14 aa, Homo sapiens
CKKKGPAAKRVKLD

SEQ ID NO: 8 - protein, 10 aa, Human immunodeficiency virus
GRKKRRQRRR

SEQ ID NO: 9 - protein, 16 aa, Drosophila melanogaster
RQIKIWFQNRRMKWKK

SEQ ID NO: 10 - protein, 26 aa, Apis florae
GIGAILKVLATGLPTLISWIKNKRKQ